(19) United States
(12) Patent Application Publication
(10) Pub. No.: US 2024/0084403 A1
(43) Pub. Date: Mar. 14, 2024

(54) Gut Microbiome as a Predictive Biomarker of Outcomes for Chimeric Antigen Receptor T-Cell Therapy

(71) Applicant: BOARD OF REGENTS, THE UNIVERSITY OF TEXAS SYSTEM, Austin, TX (US)

(72) Inventors: Neeraji Saini, Sugar Land, TX (US); Chia-Chi Chang, Houston, TX (US); Robert R. Jenq, Houston, TX (US); Sattva S. Neelapu, Bellaire, TX (US)

(73) Assignee: BOARD OF REGENTS, THE UNIVERSITY OF TEXAS SYSTEM, Austin, TX (US)

(21) Appl. No.: 18/262,338

(22) PCT Filed: Jan. 28, 2022
(86) PCT No.: PCT/US2022/014420

§ 371 Date: Jul. 20, 2023

Related U.S. Application Data
(60) Provisional application No. 63/143,665, filed on Jan. 29, 2021.

(51) Int. Cl. C12Q 1/689; A61K 35/741; A61K 39/00; A61P 35/00

(52) U.S. Cl. CPC C12Q 1/689; A61K 35/741; A61K 39/4611; A61K 39/4631; A61P 35/00; C12Q 2600/106

(57) ABSTRACT

The present disclosure concerns methods and compositions related to adoptive cell therapy and its efficacy related to the gut microbiome. In specific cases, it may be determined for an individual whether or not a chimeric antigen receptor (CAR) T-cell therapy will be effective for an individual based on their gut microbiome. An individual may be provided a composition comprising one or more particular microbe compositions based on analysis of the gut microbiome of the individual and prior to administration of CAR T-cell therapy, in specific embodiments.

This application claims priority to U.S. Provisional Patent Application Ser. No. 63/143,665, filed Jan. 29, 2021, which is incorporated by reference herein in its entirety.

### TECHNICAL FIELD

Embodiments of the disclosure concern at least the fields of cell biology, molecular biology, immunology, microbiology, and medicine.

### BACKGROUND

Considerable progress has been made in cancer therapeutics recently with targeted strategies that are efficacious and less toxic. Immunotherapy and chimeric antigen receptor (CAR) T-cells are increasingly being evaluated in a variety of tumors in the relapsed/refractory as well as frontline disease settings, predominantly in hematologic malignancies. Despite impressive outcomes in select patients, there remains significant heterogeneity in clinical response to CAR T-cells. The present disclosure provides a solution to such a need.

### BRIEF SUMMARY

The present disclosure is directed to systems, methods, and compositions related at least to making a determination of an outcome (or likelihood of an outcome) for an immunotherapy, such as adoptive cell transfer therapy. The present disclosure also is directed to systems, methods, and compositions for making a determination of toxicity (or likelihood of toxicity) for an immunotherapy, such as adoptive cell transfer therapy. The present disclosure also is directed to systems, methods, and compositions for an immunotherapy, such as adoptive cell transfer therapy. Specific embodiments of the disclosure allow for a medical practitioner to make informed decisions regarding the likelihood whether or not a therapy will be efficacious and/or toxic for an individual.

In particular embodiments, gut microbiota diversity metrics and compositions correlate with efficacy and/or toxicities associated with adoptive cell transfer therapy (such as cells that express one or more engineered antigen receptors, regardless of the types of cells that express it) directed to a cancer. The present disclosure relates to compositions and methods for predicting a subject's response to a adoptive therapy (e.g., CAR T-cell therapy), including by analyzing the intestinal microbiome of the subject. The disclosure concerns compositions and methods for determining a subject's risk for a non-efficacious CAR T-cell therapy (including partial response, stable disease, or progressive disease) and/or risk for toxicity of the CAR T-cell therapy, including by analyzing the intestinal microbiome of the subject. The present disclosure further provides therapeutic compositions and methods for treating a subject having a cancer, including to improve the efficacy and/or reduce toxicity of CAR T-cell therapy. In some cases, analysis of the gut microbiome of an individual in need of CAR T-cell therapy provides information that determines that the individual needs a particular intervention that improves the CAR T-cell therapy for the individual and/or reduces toxicity of the CAR T-cell therapy for the individual. Such intervention may include one or more probiotic compositions and/or one or more fecal transplants, as examples only.

Embodiments of the disclosure include methods of determining or predicting a therapy response for an individual, comprising the step of analyzing a microbe composition from the gut microbiome of the individual, wherein:
(a) the therapy for the individual will not be efficacious or has a risk of not being efficacious, compared to a standard or another individual, when the gut microbiome for the individual comprises, consists of, or consists essentially of one or more microbes from the Order Bacillales; and/or the species Phascolarctobacterium succinatutens; and/or Finegoldia magna; and/or Streptococcus anginosus group; and/or Akkermansia muciniphila; and/or Escherichia coli; and/or Haemophilus parainfluenzae; and/or
(b) the therapy for the individual will be efficacious or has an increased chance of being efficacious, compared to a standard or another individual, when the gut microbiome for the individual comprises, consists of, or consists essentially of one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae (such as Odoribacter splanchnicus); Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii. In specific examples of (a), the individual has cancer and the response to the therapy for the individual is or has a risk of being a partial response, stable disease, or progressive disease. In specific examples of (b), the individual has cancer (such as a solid tumor or is a hematological malignancy) and the response to the therapy for the individual is or has an increased chance of being a complete response. In specific examples of (b), the gut microbiome of the individual comprises Flavonifractor plautii.

Therapies referred to herein may be of any suitable kind, including immunotherapy, such as adoptive cell therapy that may be adoptive T-cell therapy, adoptive NK-cell therapy, and so forth. In specific embodiments, the cells of the adoptive cell therapy are modified to comprise one or more engineered antigen receptors, such as wherein an engineered antigen receptor comprises one or more chimeric antigen receptors (CAR) or one or more non-native T-cell receptors or the cells have one or more of both. In specific cases, the adoptive cell therapy comprises CAR T-cell therapy.

In some embodiments for element (b) above, the method further comprises the step of administering a therapeutically effective amount of the therapy to the individual.

In some embodiments for element (a) above, the therapy is CAR T-cell therapy and the CAR T-cell therapy is modified prior to administering to the individual to enhance efficacy of the CAR T-cell therapy. The dosage of the CAR T-cell therapy may be increased.

In any methods of the disclosure, one or more components of the CAR are altered, such as upon determination of the microbe composition from the gut microbiome of the individual in need of CAR therapy.

In specific embodiments, the individual is administered an effective amount of one or more probiotics and/or one or more fecal transplants. In specific cases, the one or more probiotics and/or one or more fecal transplants comprises, consists of, or consists essentially of one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae (such as Odoribacter splanchnicus); Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii.

In particular embodiments of methods of the disclosure, the individual is provided a therapeutically effective amount of a cancer therapy other than the adoptive cell therapy comprising cells that express an engineered antigen receptor.

Embodiments of the disclosure include methods of determining or predicting toxicity of a therapy for an individual, comprising the step of analyzing a microbe composition from the gut microbiome of the individual, wherein:
(a) the therapy will be toxic for the individual, or has a risk of being toxic for the individual, compared to a standard or another individual, when the gut microbiome comprises, consists of, or consists essentially of one or more of Species Lactobacillus rhamnosus; Species Parabacteroides goldsteinii; Species Olsenella uli; Species Fusobacterium varium; Genus Porphyrobacter; Species Dialister succinatiphilus; Species Faecalitalea cylindroides; Species Porphyrobacter sanguineus; Species Ruminiclostridium 9 unclass; Family Sphingomonadaceae; Order Sphingomonadales; and Genus Olsenella; and/or
(b) the therapy will not be toxic for the individual or has an increased likelihood of not being toxic for the individual, compared to a standard or another individual, when the gut microbiome comprises, consists of, or consists essentially of one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula. In specific embodiments, the toxicity comprises cytokine release syndrome (CRS). Determining the toxicity may be further defined as determining a grade of toxicity associated with CRS. The therapy may comprise immunotherapy, such as adoptive cell therapy, including adoptive cell therapy that comprises adoptive T-cell therapy. In some cases, the cells of the adoptive cell therapy are modified to comprise one or more engineered antigen receptors, and the engineered antigen receptor may comprise one or more chimeric antigen receptors (CAR) or one or more non-native T-cell receptors or the cells have one or more of both. In specific embodiments, the adoptive cell therapy comprises CAR T-cell therapy. In certain methods of the disclosure, the method element of (b) may further comprise the step of administering a therapeutically effective amount of the therapy to the individual. In some cases of the method, in element (a) the therapy is CAR T-cell therapy and the CAR T-cell therapy is modified prior to administering to the individual to reduce toxicity of the CAR T-cell therapy. The dosage of the CAR T-cell therapy may be decreased and/or one or more components of the CAR are altered.

In particular embodiments, the individual is administered an effective amount of one or more probiotics and/or one or more fecal transplants. The one or more probiotics and/or one or more fecal transplants may comprise, consist of, or consist essentially of one or more microbes from Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula. In some cases, the individual is provided a therapeutically effective amount of another cancer therapy.

Embodiments of the disclosure may include a method of determining the likelihood of an individual receiving an immunotherapy to have a particular grade of immune effector cell-associated neurotoxicity syndrome (ICANS) toxicity following receipt of the immunotherapy, comprising the step of analyzing the gut microbiome of the individual, wherein:
(a) the individual is likely of having or at risk for having Grade 3 or 4 of ICANS toxicity when the gut microbiome comprises, consists of, or consists essentially of one or more of Species [Clostridium] lavalense; Genus Cuneatibacter; Family Clostridiales vadin BB60 group; Genus Clostridiales vadin BB60 group; Species Clostridiales vadin BB60 group ge unclass; Species [Clostridium] hylemonae; Species Anaerotruncus rubiinfantis; Genus Ruminococcaceae UCG 004; Species Eisenbergiella massiliensis; Genus Hydrogenoanaerobacterium; Genus Ruminococcaceae UCG 008; Species Intestinibacillus massiliensis; Genus Raoultibacter; Species Monoglobus pectinilyticus; Family Bacillaceae; Species Pseudomonas aeruginosa; Species Bacillus hisashii; Species Caecibacter massiliensis; and Species Prevotella multisaccharivorax; and/or
(b) the individual is likely of having or at risk for having Grade 1 or 2 of ICANS toxicity when the gut microbiome comprises, consists of, or consists essentially of one or more of Genus Corynebacterium; Species Lactobacillus sakei; Species Veillonella dispar; Species Streptococcus salivarius; and Species Coprococcus eutactus. The therapy may comprise immunotherapy, such as adoptive cell therapy, including adoptive cell therapy that comprises adoptive T-cell therapy. The cells of the adoptive cell therapy may be modified to comprise one or more engineered antigen receptors, and the engineered antigen receptor may comprise one or more chimeric antigen receptors (CAR) or one or more non-native T-cell receptors or the cells have one or more of both. The adoptive cell therapy may comprise CAR T-cell therapy.

In the method element of (b), the method may further comprise the step of administering a therapeutically effective amount of the immunotherapy to the individual. In the method element (a), following the analysis the immunotherapy may be modified to reduce the risk of ICANS prior to delivery to the individual. In the method element (a), the therapy is CAR T-cell therapy and the CAR T-cell therapy is modified prior to administering to the individual to reduce toxicity of the CAR T-cell therapy. In some cases the dosage of the CAR T-cell therapy is decreased and/or one or more components of the CAR are altered. In specific embodiments of the method, the individual is administered an effective amount of one or more probiotics and/or one or more fecal transplants. The one or more probiotics and/or one or more fecal transplants may comprise, consist of, or consist essentially of one or more of Genus Corynebacterium; Species Lactobacillus sakei; Species Veillonella dispar; Species Streptococcus salivarius; and Species Coprococcus eutactus. The individual may be provided a therapeutically effective amount of another cancer therapy.

Embodiments of the disclosure include methods of treating cancer in an individual, comprising the step of providing a therapeutically effective amount of an immunotherapy to the individual when:
(a) the gut microbiome of the individual comprises, consists of, or consists essentially of one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; SpeciesAnaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) the gut microbiome of the individual comprises, consists of, or consists essentially of one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) the gut microbiome of the individual comprises, consists of, or consists essentially of one or more of Genus Corynebacterium; Species Lactobacillus sakei; Species Veillonella dispar; Species Streptococcus salivarius; and Species Coprococcus eutactus. In some embodiments, the immunotherapy comprises adoptive cell therapy, such as adoptive T-cell therapy. The cells of the adoptive cell therapy may be modified to comprise one or more engineered antigen receptors, and the engineered antigen receptor may comprise one or more chimeric antigen receptors (CAR) or one or more non-native T-cell receptors or the cells have one or more of both. The adoptive cell therapy may comprise CAR T-cell therapy. In some embodiments, the individual is administered an effective amount of one or more probiotics and/or one or more fecal transplants, and in some cases the one or more probiotics and/or one or more fecal transplants comprises, consists of, or consists essentially of one or more microbes of any one or more of (a), (b), and/or (c):
(a) one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) one or more of Genus Corynebacterium; Species Lactobacillus sakei; Species Veillonella dispar; Species Streptococcus salivarius; and Species Coprococcus eutactus. In some embodiments, the individual is provided a therapeutically effective amount of another cancer therapy.

Embodiments of the disclosure include methods of determining a therapy outcome for an individual in need of adoptive cell therapy, comprising the step of analyzing the gut microbiome for diversity of microbes therein, wherein when the gut microbiome of the individual has high diversity, the individual has an increased likelihood of efficacious adoptive cell therapy, compared to an individual that lacks high diversity of the gut microbiome. In specific embodiments, when the individual has a low diversity of microbes in the gut microbiome, the individual is provided an effective amount of one or more fecal transplantations and/or one or more probiotic compositions. The diversity of the gut microbiome may or may not be determined by Inverse Simpson Index (ISI). In some cases, when the diversity of the individual's gut microbiome is in the highest tertile as determined by ISI, the individual has an increased likelihood of efficacious adoptive cell therapy. In some cases, the identity of one or more microbes in the microbiome is determined by shotgun sequencing of the genome of the one or more microbes. The identity of one or more microbes in the microbiome may be determined by directed sequencing of the genome of the one or more microbes, and the directed sequencing may be of 16S rRNA of the one or more microbes. In specific embodiments, when the diversity of the individual's gut microbiome is in the highest tertile as determined by ISI, the individual is administered an effective amount of the adoptive cell therapy, such as CAR T-cell therapy. In some embodiments, when the diversity of the individual's gut microbiome is not in the highest tertile as determined by ISI, the individual is not administered an effective amount of the adoptive cell therapy. In some cases, the adoptive cell therapy is CAR T-cell therapy and when the diversity of the individual's gut microbiome is not in the highest tertile as determined by ISI, the CAR T-cell therapy is modified, such as the dosage of the CAR T-cell therapy being decreased and/or one or more components of the CAR are altered. In specific embodiments, the individual is administered an effective amount of one or more probiotics and/or one or more fecal transplants. The one or more probiotics and/or one or more fecal transplants may comprise, consist of, or consist essentially of one or more microbes of any one or more of (a), (b), and/or (c):
(a) one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) one or more of Genus Corynebacterium; Species Lactobacillus sakei; Species Veillonella dispar; Species Streptococcus salivarius; and Species Coprococcus eutactus.

Embodiments of the disclosure include a probiotic composition and/or fecal transplant composition, comprising, consisting of, or consisting essentially of one or more microbes of any one or more of (a), (b), and/or (c):
(a) one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae (such as Odoribacter splanchnicus); Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) one or more of Genus Corynebacterium; Species Lactobacillus sakei; Species Veillonella dispar; Species Streptococcus salivarius; and Species Coprococcus eutactus.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary of Invention, Brief Description of the Drawings, Detailed Description, and Claims.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1C. Association of gut microbiome with clinical outcome in relapsed/refractory large B-cell lymphoma (r/r LBCL) patients treated with CAR T-cell therapy. FIG. 1A. Inverse Simpson index (alpha diversity; ISI) of the gut microbiome in patients with ongoing complete response (CR) versus no complete response (NoCR). FIG. 1B. Kaplan-Meier plot of pFS by ISI tertiles (high n=11, intermediate n=11, low n=11). FIG. 1C. Kaplan-Meier plot of OS by ISI tertiles.

FIG. 2. Linear discriminant analysis showing differential abundance of gut or microbiome species associating with ongoing CR at 3 months.

FIG. 3. Heatmap revealing the difference in bacterial composition associating with responses.

FIG. 4. Linear discriminant analysis showing differential microbial composition associating with CRS toxicity (grade 0-1 vs. ≥2).

FIG. 5. Heatmap revealing the difference in bacterial composition associating with Cytokine release syndrome (CRS) toxicity (grade 0-1 vs. ≥2).

FIG. 6. Linear discriminant analysis showing differential microbial composition associating with Immune effector cell-associated neurotoxicity syndrome (ICANS) toxicity (grade 0-2 vs. ≥3).

FIG. 7 demonstrates taxonomy results that indicate whether or not the presence of the bacteria favors disease progression.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Examples of Definitions

In keeping with long-standing patent law convention, the words “a” and “an” when used in the present specification in concert with the word comprising, including the claims, denote “one or more.” As used in the specification and claims, the singular form “a”, “an”, and “the” include plural references unless the context clearly dictates otherwise. For example, the term “a nucleic acid” includes a plurality of nucleic acids, including mixtures thereof. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “gut” as used herein refers to at least part of the digestive tract, including at least the stomach, small intestine, and large intestine.

The term “microbe” as used herein refers to a microorganism, such as bacteria, fungi, virus, protozoa, algae, amoebas, slime molds, or a combination thereof. For any bacteria encompassed in the present disclosure, the disclosure includes any strains thereof. In the present disclosure, when a Genus is specifically referred to, any species within the genus may also be incorporated by reference, such as incorporated by reference into a list. For example, if Genus Corynebacterium is referred to, then any species within the Genus Corynebacterium is encompassed herein.

The term “microbiome,” as used herein, generally refers to a collection of microorganisms (that also may be referred to as microbes), such as bacteria, fungi, viruses, protozoa, algae, amoebas, and/or slime molds within a community in a host, including within a particular location and/or tissue and/or organ of a host, such as the gut.

The term “probiotic” as used herein refers to one or more microbes introduced into the body for beneficial qualities.

The term “sample,” as used herein, generally refers to a biological sample, including from any region in the body, such as the gut. The sample may be taken from tissue or cells or from the gut environment. In some examples, the sample may comprise, or be derived from, any part of the body tissues, including a tissue biopsy, stool, blood, lung tissue, tumors, or a combination thereof. The sample may have been isolated from the source prior to collection. In some examples, the sample is isolated from its primary source (cells, tissue, bodily fluids such as blood, environmental samples, etc.) during sample preparation. The sample may or may not be purified or otherwise enriched from its primary source. In some embodiments, the primary source is homogenized prior to further processing. The sample may be filtered or centrifuged to remove undesired material. The sample may also be purified or enriched for particular compositions therein, such as particular microbes. The sample may contain tissues or cells that are intact, fragmented, or partially degraded.

The term “subject,” as used herein, generally refers to an individual having a biological sample that is undergoing processing or analysis and, in some embodiments, has a gut microbiome associated therewith. A subject can be an animal and can be the desired recipient of an immunotherapy, such as adoptive cell therapy. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more cancers. The subject may be asymptomatic. The term “individual” may be used interchangeably, in at least some embodiments. The “subject” or “individual”, as used herein, may or may not be housed in a medical facility and may or may not be treated as an outpatient of a medical facility. The individual may be receiving one or more medical compositions via the internet. An individual may comprise any age of a human or non-human animal and therefore includes both adult and juveniles (e.g., children) and infants and includes in utero individuals. A subject may or may not have a need for medical treatment; an individual may voluntarily or involuntarily be part of experimentation whether clinical or in support of basic science studies. The individual may be of any gender, race, or age.

### II. Embodiments of the Disclosure

The gut microbiome has emerged as one of the key host factors that may be relevant to responses to immunotherapy. Several recent human studies evaluating immunotherapy strategies such as immune checkpoint inhibitor therapy showed a significantly superior response and survival in patients with the more diverse gut microbiome. Currently, it is unknown if gut microbiota modulates anti-tumor responses to adoptive cell therapy such as CAR T-cells. In this disclosure, it is shown that gut microbiota diversity and particular gut microbiome compositions for an individual correlate with efficacy and toxicities associated with CAR-T therapy (as an example of adoptive cell therapy) for a cancer.

The present disclosure relates to methods and compositions for the treatment of cancer by modulating the microbiome to enhance the efficacy of CAR-T therapy. The present disclosure also relates to microbiome diversity metrics and bacterial abundance as a biomarker for the prediction of efficacy and toxicities related to CAR-T therapy.

The results encompassed herein are associated with particular compositions of the gut bacterium that can associate with efficacy and toxicity. In particular embodiments, the gut microbiome is analyzed for an individual in need of an immunotherapy, and that analysis may occur by any suitable method, including Shotgun sequencing that can provide in-depth reads or the standard 16S rRNA sequencing, for example. For microbiome studies, shotgun sequencing can identify and profile bacteria, fungi, viruses and many other types of microorganisms at the same time.

In some embodiments, probiotics or fecal microbiota transplantation (and, in some cases, associated metabolites/proteins) may be utilized to modulate responses to CAR-T therapy to enhance their efficacy and/or safety. In such cases, prior analysis of the gut microbiome allows determination of which individuals would need the probiotic or fecal transplant, allowing for informed clinical decision making.

### A. Examples of Methods of Use

Methods of the disclosure allow for improvement of targeted therapeutic strategies or determination of efficacy (e.g., improves at least one symptom) for targeted therapeutic strategies, including immunotherapy such as adoptive cell transfer. Although the adoptive cell transfer can be of any kind, in specific embodiments the adoptive cell transfer concerns cells that have been engineered by the hand of man to express one or more engineered antigen receptors. The antigen receptors may be engineered to specifically target an antigen that is associated with a deleterious medical condition, such as a cancer antigen associated with one or more specific cancers. The cancer may or may not be relapsed or refractory. The cancer may be solid tumors or hematological malignancies. The cancer may be of any stage or type or tissue of origin. The cancer may or may not be metastatic. The cancer may or may not be resistant to one or more types of therapies.

In specific embodiments, a sample from an individual is analyzed to determine the likelihood of efficacy for an immunotherapy, such as adoptive cell transfer, for the individual. Although the adoptive cell transfer may comprise cells that express one or more engineered antigen receptors, in specific embodiments the engineered antigen receptor is a chimeric antigen receptor (CAR) and/or non-native T-cell receptor. In specific cases, the adoptive cell therapy comprises adoptive cell therapy where the cells express one or more CARs, and in certain cases the cells are T cells, NK cells, NKT cells, gamma-delta T cells, macrophages, B cells, or a mixture of these.

Methods of the disclosure includes methods where a response to adoptive cell transfer therapy is enhanced to increase the likelihood of the adoptive cell transfer being efficacious. In some cases, the method is employed for an individual where it is uncertain whether or not an adoptive cell transfer will be efficacious, whereas in other cases the method is employed for an individual where it is known that the adoptive cell transfer may not be efficacious for the individual. In other cases, it has been determined that the adoptive cell transfer should be efficacious for the individual, but the methods of the disclosure are still employed as a routine matter or in the general therapeutic interest of the individual.

The present disclosure encompasses methods and compositions related to the gut microbiome of an individual that has cancer, or that is suspected of having cancer, and is in need of intervention with immunotherapy, including adoptive cell transfer.

In some embodiments, the likelihood of toxicity from an adoptive cell transfer for an individual is determined based on the gut microbiome, including prior to delivery of the adoptive cell transfer to the individual. In such cases, as a result the adoptive cell transfer may not be given to the individual, or the adoptive cell transfer is altered to avoid toxicity for the individual, or the individual is given one or more other agents to reduce or eliminate the toxicity of the immunotherapy for the individual (whether or not the agent(s) is given at the same time as the immunotherapy, before the immunotherapy, and/or after the immunotherapy). For example, the individual may be given an effective amount of an agent that activates a suicide gene in cells of adoptive cell transfer. The one or more agents may comprise one or more probiotics and/or one or more fecal transplantations. In some embodiments, the direct or indirect cause of toxicity from an adoptive cell transfer already given to an individual is determined based on the gut microbiome following delivery of the adoptive cell transfer.

The disclosure encompasses methods and compositions for modulating the gut microbiome of an individual to enhance efficacy of adoptive cell transfer therapy. The modulation may or may not be as a result of analysis of the gut microbiome prior to delivery of the adoptive cell transfer therapy to the individual. In some cases, the modulation is a result of analysis of the gut microbiome prior to delivery of the adoptive cell transfer therapy, and the outcome of the analysis determined the nature of the resultant modulation of the gut microbiome. For example, the modulation may comprise providing an effective amount of a composition comprising one or more microbes that were determined to be deficient in the gut microbiome of the individual. In some cases, the modulation may comprise providing an effective amount of one or more antibiotics that would reduce levels of one or more microbes that were determined to be excessive in the gut microbiome of an individual. In some cases, both a deficiency in the gut microbiome and an excess in the gut microbiome are both handled prior to delivery of the adoptive cell transfer therapy. In particular embodiments, such actions improve the efficacy of the adoptive cell transfer therapy and/or reduce toxicity of the adoptive cell transfer therapy.

In particular embodiments, the disclosure concerns methods of predicting whether or not adoptive cell transfer therapy will be efficacious or toxic for an individual based on analyzing one or more of the following biomarkers: (1) diversity of the gut microbiome; and/or (2) analyzing the level of one or more specific microbes in the gut microbiome. In some embodiments, the disclosure concerns methods of determining the likelihood of an adoptive cell transfer therapy to be efficacioius or toxic in an individual, such as when compared to a standard or an individual with a different microbiome. Such analysis of (1) and/or (2) above results in a determination whether or how best to utilize the adoptive cell transfer therapy. For example, when the analysis of the gut microbiome indicates that the adoptive cell transfer therapy will be efficacious for an individual (or there is an increased chance that it will be efficacious), the individual may then receive a therapeutically effective amount of the adoptive cell transfer therapy. When the analysis of the gut microbiome predicts that the adoptive cell transfer therapy will not be efficacious for the individual (or there is an increased risk that it will not be efficacious), the individual may then not receive the adoptive cell transfer therapy, or the regimen and/or composition of the adoptive cell transfer therapy may itself be modified, or the individual may be given an effective amount of one or more microbes that would then alter the gut microbiome to improve the efficacy of the adoptive cell transfer therapy. When the analysis of the gut microbiome predicts that the adoptive cell transfer therapy will be toxic for the individual (or there is an increased chance that it will be toxic compared to an individual with a different gut microbiome), the individual may then not receive the adoptive cell transfer therapy, or the regimen and/or composition of the adoptive cell transfer therapy may itself be modified, or the individual may be given an effective amount of one or more microbes that would then alter the gut microbiome to reduce toxicity of the adoptive cell transfer therapy. In any of these predicted or at risk cases, the individual may receive an effective amount of one or more probiotics and/or one or more fecal transplantations to increase the likelihood of the therapy being efficacious and/or non-toxic to the individual.

In some embodiments, following analysis of a gut microbiome of an individual, it is determined that an individual is in need of a modification of the gut microbiome, including prior to being administered a particular therapy, such as an immunotherapy, including adoptive cell transfer therapy. In some cases, the modification comprises administering to the individual an effective amount of one or more compositions that modify the gut microbiome such that the presence of one or more microbes and/or the level of one or more microbes are modified. In some embodiments, the individual is provided a composition such as a probiotic that comprises one or more microbes of which the individual may be considered to be deficient, including upon analysis of the gut microbiome. The composition may comprise one or more microbes that are deficient in the individual's gut microbiome, and then following this administration (whether it be by one or more administrations), the individual's gut microbiome is then modified to a sufficient level such that the individual can then be the recipient of the particular therapy, including the particular adoptive cell therapy. In specific cases, an individual is determined to be deficient in one or more microbes, and the individual is provided an effective amount of one or more compositions comprising the one or more microbes (in one or more administrations) upon which the individual is provided an effective amount of a particular adoptive cell therapy such that the adoptive cell therapy (as a result of the modification of the gut microbiome) is more efficacious compared to if the individual did not receive the composition(s). In some situations, alternatively or additionally, following the administration of the individual is able to receive the one or more compositions comprising the one or more microbes (in one or more administrations) with a reduced toxicity compared to if the individual did not receive the compositions(s).

The disclosure encompasses the determination or prediction whether an adoptive cell transfer therapy such as CAR T-cell therapy, would be therapeutic in an individual by analyzing the microbiome and, if the analysis determines the individual is in need of modification of the gut microbiome, the individual is provided an effective amount of a composition that addresses the deficiency of the microbiome. As one example, the individual may be provided an effective amount of a composition that (1) comprises one or more microbes present at insufficient levels in the individual's gut microbiome; (2) comprises one or more agents to reduce the level of excessive levels of one or more microbes in the gut microbiome (such as an antibiotic); or (3) both. The composition of (1) may or may not be a probiotic or may or may not be fecal transplantation.

In specific embodiments, the present disclosure encompasses the customizing of one or more compositions such that an individual receives one or more microbes that are deficient in their gut microbiome, as compared to a control or standard or normal individual (such as an individual not having cancer or an individual determined to have a highly diverse gut microbiome or that has specific microbe(s)). The customization may be done following analysis of the individual's gut microbiome, or the content of the composition may be standardized compared to deficiencies consistent in the general population, in at least some cases. In specific embodiments, a composition such as a probiotic may be formulated following analysis of an individual's gut microbiome, whereas in other cases a probiotic may be given to individuals that may have differences in their gut microbiome but that have common deficiencies among them. A deficiency may be an insufficient level of one or more beneficial microbes in the gut, an excessive level of one or more non-beneficial microbes in the gut, a gut microbiome that lacks a certain level of diversity, or a combination thereof.

1. Overall Microbial Diversity

In particular embodiments, the gut microbiome of an individual is analyzed for the overall diversity of its microbes, irrespective of which microbes are actually present and/or absent. In particular cases, an individual having a highly diverse gut microbiome has an increased chance of efficacious adoptive cell transfer therapy, such as improving one or more symptoms or having a complete response. In particular cases, an individual having a low diversity in the gut microbiome has a reduced chance of efficacious adoptive cell transfer therapy. In particular cases, an individual having a highly diverse gut microbiome has a reduced chance of toxicities directly or indirectly related to the adoptive cell transfer therapy. In particular cases, an individual having a low diversity in the gut microbiome has an increased chance of toxicities directly or indirectly related to the adoptive cell transfer therapy. In at least some cases, an individual with a highly diverse gut microbiome may be expected to have progression-free survival after a particular period of time (e.g., 1, 2, 3, 4, 5, 6, 7, 8, 9, 10, 11, or 12, 13, 14, 15, 16, 17, 18, or more months following initial treatment with the adoptive cell transfer therapy).

The overall diversity of the gut microbiome may be measured in any suitable manner; in specific aspects there are multiple ways to quantify the microbiome, including microbial alpha diversity, such as Shannon index, observed outcome, etc. Measurements of diversity have historically relied on the species as the fundamental unit of analysis. Diversity within a given community (alpha diversity) is usually characterized using the total number of species (species richness), the relative abundancies of the species (species evenness), or indices that combine these two dimensions. In some embodiments, the diversity is measured as a function of Inverse Simpson Index (ISI) to calculate microbial alpha diversity. Alternatives include observed OUTs-count of different species/OTUs in each sample, Chao1 index-esimate diversity from abundance data, and Shannon diversity. Quantitative species-based measures, such as ISI are often used to summarize and compare the microbiome alpha diversity in different communities. The diversity of the gut microbiome in the individual may be compared to a standard produced based on ISI analysis, wherein if the individual's gut microbiome value is in the highest tertile (for example, a cutoff for ISI of 10, 11, 12, 13, 14, 15, 16, 17, 18, 19, or 20), the individual will have an efficacious adoptive cell transfer therapy, such as may be measured by progression-free survival or complete response, for example. In particular embodiments, if the individual's overall gut microbiome value is in the lowest tertile, the individual is at risk for having a poor outcome with the adoptive cell transfer therapy, such as having no response, poor response, partial response, stable disease, or progressive disease.

In particular embodiments, the standard produced based on ISI analysis is in part a function of the number of sequencing reads and/or number of individuals analyzed for the standard. The standard may be produced based upon known individuals and upon knowledge of which of the individuals had complete response or lacked a complete response. The tertiles for the ISI may be stratified based upon the number of individuals having CR vs. no CR after a specific time as a function of the diversity of their gut microbiome. In a specific example, a high tertile is considered to be 16 or greater and identifies the individual as expected at least to have progression-free survival and CR of 3 months. In a specific example, in 1.5 million reads, an ISI of 16 was predictive of high diversity and response. In specific aspects, the average raed counts per sample is around 49,000, and the average of ISI in this cohort is 13.8, whereas the median of ISI is 13.4. Therefore, in some embodiments, for response prediction with an average of 49,000 reads, an individual with an ISI score of 16 will have a higher chance of obtaining response from CAR T-cell therapy.

In embodiments wherein the individual is determined to have a gut microbiome that is of low diversity, the individual may or may not receive the adoptive cell therapy. In cases wherein the individual receives the therapy despite having a gut microbiome of low diversity, it may be because the individual has received an effective amount of one or more agents that improve the diversity of the gut microbiome, such as fecal transplant and/or probiotic composition(s). When an individual is determined to have a gut microbiome of high diversity, they may receive a therapeutically effective amount of the adoptive cell therapy.

2. No Complete Response

Particular embodiments of the disclosure concern analysis of gut microbiomes in individuals who are in need of treatment with immunotherapy, such as CAR T-cell therapy. Specifically, individuals in need of CAR T-cell therapy have their gut microbiomes analyzed prior to the therapy. In at least certain embodiments, the presence of one or more specific microbes, the absence of one or more specific microbes, or a combination thereof, identifies the type of response to the therapy the individual will have or has an increased likelihood of having when compared to an individual with a different gut microbiome (such as an individual that has differences in the the presence of one or more specific microbes and/or the absence of one or more specific microbes when compared to the individual in need of the therapy).

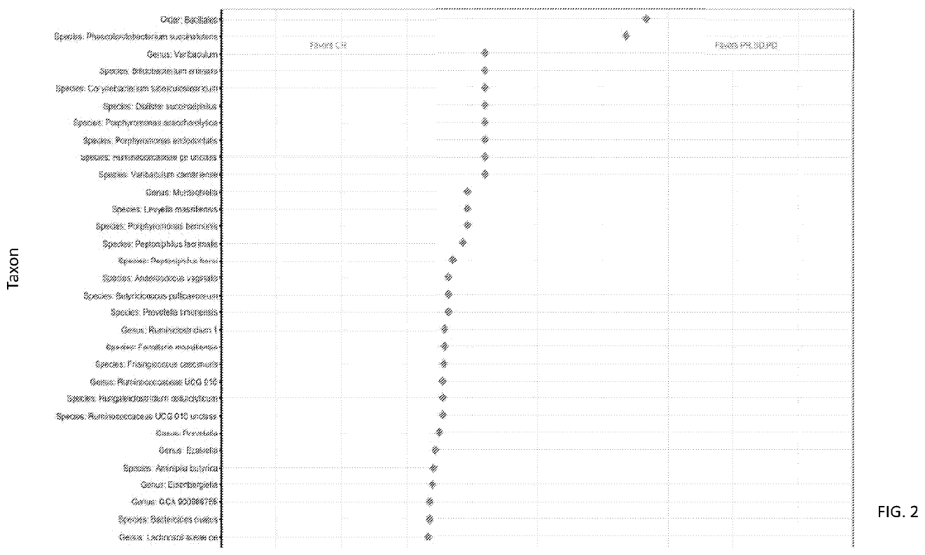

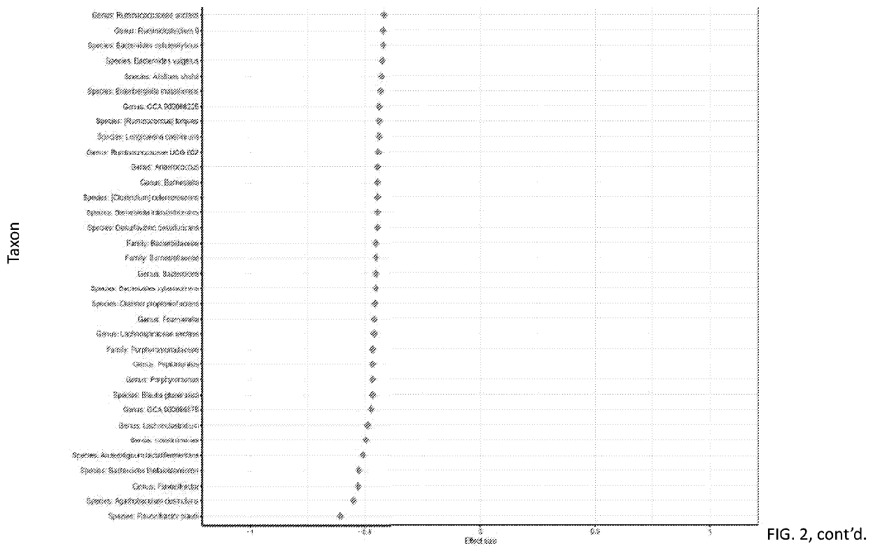

As addressed in FIG. 2, individuals at 3 months after therapy who had a complete response to the therapy had a different initial gut microbiome composition than those who had partial response, PR; stable disease, SD; or progressive disease, PD. In specific embodiments, those individuals who had microbes from the Order Bacillales and/or who had Species Phascolarctobacterium succinatutens had, or had an increased risk of having, PR, SD, or PD. In cases wherein the individual has an increased risk of having PR, SD, or PD, such a risk of having PR, SD, or PD may be increased compared to individuals that did not have such microbe(s). In specific embodiments, the individual's gut microbiome has 1, 2, 3, 4, 5, 6, 7, 8, 9, 10, 11, 12, 13, 14, 15, 16, 17, 18, 19, 20, or more microbes from any family in the order Bacillales, including from any species in the genera Bacillus, Listeria and Staphylococcus, as examples only. In cases wherein an individual is determined to have one or more bacteria from the Order Bacillales and/or determined to have Phascolarctobacterium succinatutens, as a result of the determination the individual may or may not receive the CAR T-cell therapy. In cases wherein the individual still receives the CAR T-cell therapy despite having one or more of the aforementioned microbes, the individual may receive one or more additional cancer therapies than the CAR T-cell therapy, such as surgery, chemotherapy, drug therapy, radiation, hormone therapy, or a combination thereof. In some cases when the individual will still receive a CAR T-cell therapy despite having one or more of the aforementioned microbes, the CAR T-cell therapy may be given in a different dosage (such as greater) and/or may be modified to be able to overcome the risk of the individual having PR, SD, or PD. For example, the target antigen of the CAR may be changed; additional or different costimulatory domains may be utilized in the CAR; an additional CAR targeting an additional target antigen may be utilized in the cells; one or more cytokines (such as IL-15) may be utilized with the CAR T-cell therapy, and so forth. In specific embodiments, additional hinge and transmembrane domains of CAR-T therapy may be utilized. CAR-T secreting antibodies or any protein may also be utilized. The individual may be given one or more antibiotics, including that target one or more microbes that over come any deficiencies in the individual's gut microbiome, such as target one or more microbes of Order Bacillales and/or Species Phascolarctobacterium succinatutens.

With respect to the Order Bacillales, representative families include Alicyclobacillaceae; Bacillaceae; Listeriaceae; Paenibacillaceae; Pasteuriaceae; Planococcaceae; Sporolactobacillaceae; Staphylococcaceae; and Thermoactinomycetaceae; representative genera in the Order Bacillales include Bacillus, Listeria and Staphylococcus.

3. Complete Response

As addressed in FIG. 2, individuals at 3 months after therapy who had a complete response to the therapy had a particular gut microbiome composition. With respect to the method of what database was used to match to the sequencing results and generate OUTs, the following was utilized: The amplicon pool was purified with QIAquick gel extraction kit (Qiagen) and sequenced on the Illumina Miseq sequencer platform using 2×250 bp paired-end protocol. After sequencing, paired-end reads were de-multiplexed by QIIME and then merged and dereplicated for chimeras using VSEARCH. UNOISE 3 command algorithm was used to perform denoising of reads (R. C. Edgar. bioRxiv, 081257, 2016). Operational taxonomic units (OUTs) were classified using Mothur method with the Silva database version 138. For differential taxa-based univariate analysis, abundant microbiome taxa at species, genus, family, class, and order levels were analyzed using Mann-Whitney U-test after logit transformation. The detailed computational pipeline of analysis has been previously described (Yinghong Wang et al. Nat Med. 2018). For exploratory analyses, p values have not been adjusted for multiple comparisons.

With SILVA release 138, Genome Taxonomy Database (GTDB) has been adopted (Parks 2018). As a consequence of efforts, in specific cases the following groups were prone to significant adaptations: Archaea, Enterobacterales, Deltaproteobacteria, Firmicutes, Clostridia. Betaproteobacteriales (formerly known as Betaproteobacteria) is now Burkholderiales, an order of Gammaproteobacteria. Epsilonproteobacteria vanishes within a new phylum Campilobacterota. Tenericutes are gone, they are now all part of Bacilli, inside Firmicutes. Additionally, every sequence in the SILVA datasets carries the EMBL-EBI/ENA taxonomy assignment. Where available, the RDP and GTDB taxonomies are added for comparison. In releases <138 also the greengenes taxonomy was added. The inventors used SILVA for bacterial classification.

In specific aspects, those individuals who had one or more microbes from the following list had an increased chance of having complete response, including an increased chance when compared to individuals that did not have one or more microbes from the following list:
Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species Ruminococcus torques; Species Longicatena caecimuris; Genus Ruminococcaceae UCG 002; Genus Anaerococcus; Genus Barnesiella; Species Clostridium celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae (such as Odoribacter splanchnicus); Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; Species Flavonifractor plautii. In specific embodiments, the individual's gut microbiome has 1, 2, 3, 4, 5, 6, 7, 8, 9, 10, 11, 12, 13, 14, 15, 16, 17, 18, 19, 20, or more of the aforementioned microbes.

In particular embodiments, when an individual is determined to have one or more of the aforementioned microbes (and in some cases the majority of or all of the microbes), the individual will have or has an increased chance of having complete response compared to an individual that does not have the corresponding microbes. In specific embodiments, when an individual is determined to have a particular number of the aforementioned microbes, such as at least 10, 15, 20, 25, 30, 35, 40, 45, 50, 55, 60, 65, 70, 75, 80, 85, 90, 95, or greater than 95% of the aforementioned microbes, the individual will have or has an increased chance of having complete response compared to an individual that does not have the same or similar number of corresponding microbes.

In particular embodiments, when an individual is determined to have one or more of the aforementioned microbes (and in some cases the majority of or all of the microbes, or a percentage as noted above) in the Complete Response list, the individual may be given a therapeutically effective amount of the CAR T-cell therapy. The individual may or may not be given an additional cancer therapy. Despite the likelihood tha the individual will have effective CAR T-cell therapy based on the gut composition, the individual may still be given one or more probiotic compositions, including that comprise one or more microbes that enhance the individual's gut microbiome.

Below, particular species are indicated from Family and Genera referred to in the Complete Response list above.

With respect to the Genus Bacillus, representative species include B. acidiceler; B. acidicola; B. acidiproducens; B. acidocaldarius; B. acidoterrestris; B. aeolius; B. aerius; B. aerophilus; B. agaradhaerens; B. agri; B. aidingensis; B. akibai; B. alcalophilus; B. algicola; B. alginolyticus; B. alkalidiazotrophicus; B. alkalinitrilicus; B. alkalisediminis; B. alkalitelluris; B. altitudinis; B. alveayuensis; B. alvei; B. amyloliquefaciens; B. aminovorans; B. amylolyticus; B. andreesenii; B. aneurinilyticus; B. anthracis; B. aquimaris; B. arenosi; B. arseniciselenatis; B. arsenicus; B. aurantiacus; B. arvi; B. aryabhattai; B. asahii; B. atrophaeus; B. axarquiensis; B. azotofixans; B. azotoformans; B. badius; B. barbaricus; B. bataviensis; B. beijingensis; B. benzoevorans; B. beringensis; B. berkeleyi; B. beveridgei; B. bogoriensis; B. boroniphilus; B. borstelensis; B. brevis Migula; B. butanolivorans; B. canaveralius; B. carboniphilus; B. cecembensis; B. cellulosilyticus; B. centrosporus; B. cereus; B. chagannorensis; B. chitinolyticus; B. chondroitinus; B. choshinensis; B. chungangensis; B. cibi; B. circulans; B. clarkia; B. clausii; B; coagulans; B. coahuilensis; B. cohnii; B. composti; B. curdlanolyticus; B. cycloheptanicus; B. cytotoxicus; B. daliensis; B. decisifrondis; B. decolorationis; B. deserti; B. dipsosauri; B. drentensis; B. edaphicus; B. ehimensis; B. eiseniae; B. enclensis; B. endophyticus; B. endoradicis; B. farraginis; B. fastidiosus; B. fengqiuensis; B. firmus; B. flexus; B. foraminis; B. fordii; B. formosus; B. fortis; B. fumarioli; B. funiculus; B. fusiformis; B. galactophilus; B. galactosidilyticus; B. galliciensis; B. gelatini; B. gibsonii; B. ginseng; B. ginsengihumi; B. ginsengisoli; B. glucanolyticus; B. gordonae; B. gottheilii; B. graminis; B. halmapalus; B. haloalkaliphilus; B. halochares; B. halodenitrificans; B. halodurans; B. halophilus; B. halosaccharovorans; B. hemicellulosilyticus; B. hemicentroti; B. herbersteinensis; B. horikoshii; B. horneckiae; B. horti; B. huizhouensis; B. humi; B. hwajinpoensis; B. idriensis; B. indicus; B. infantis; B. infernus; B. insolitus; B. invictae; B. iranensis; B. isabeliae; B. isronensis; B. jeotgali; B. kaustophilus; B. kobensis; B. kochii; B. kokeshiiformis; B. koreensis; B. korlensis; B. kribbensis; B. krulwichiae; B. laevolacticus; B. larvae; B. laterosporus; B. lautus; B. lehensis; B. lentimorbus; B. lentus; B. licheniformis; B. ligniniphilus; B. litoralis; B. locisalis; B. luciferensis; B. luteolus; B. luteus; B. macauensis; B. macerans; B. macquariensis; B. macyae; B. malacitensis; B. mannanilyticus; B. marisflavi; B. marismortui; B. marmarensis; B. massiliensis; B. megaterium; B. mesonae; B. methanolicus; B. methylotrophicus; B. migulanus; B. mojavensis; B. mucilaginosus; B. muralis; B. murimartini; B. mycoides; B. naganoensis; B. nanhaiensis; B. nanhaiisediminis; B. nealsonii; B. neidei; B. neizhouensis; B. niabensis; B. niacini; B. novalis; B. oceanisediminis; B. odysseyi; B. okhensis; B. okuhidensis; B. oleronius; B. oryzaecorticis; B. oshimensis; B. pabuli; B. pakistanensis; B. pallidus; B. pallidus; B. panacisoli; B. panaciterrae; B. pantothenticus; B. parabrevis; B. paraflexus; B. pasteurii; B. patagoniensis; B. peoriae; B. persepolensis; B. persicus; B. pervagus; B. plakortidis; B. pocheonensis; B. polygoni; B. polymyxa; B. popilliae; B. pseudalcalophilus; B. pseudofirmus; B. pseudomycoides; B. psychrodurans; B. psychrophilus; B. psychrosaccharolyticus; B. psychrotolerans; B. pulvifaciens; B. pumilus; B. purgationiresistens; B. pycnus; B. qingdaonensis; B. qingshengii; B. reuszeri; B. rhizosphaerae; B. rigui; B. ruris; B. safensis; B. salaries; B. salexigens; B. saliphilus; B. schlegelii; B. sediminis; B. selenatarsenatis; B. selenitireducens; B. seohaeanensis; B. shacheensis; B. shackletonii; B. siamensis; B. silvestris; B. simplex; B. spiralis; B. smithii; B. soli; B. solimangrovi; B. solisalsi; B. songklensis; B. sonorensis; B. sphaericus; B. sporothermodurans; B. stearothermophilus; B. stratosphericus; B. subterraneus; B. subtilis; B. taeanensis; B. tequilensis; B. thermantarcticus; B. thermoaerophilus; B. thermoamylovorans; B. thermocatenulatus; B. thermocloacae; B. thermocopriae; B. thermodenitrificans; B. thermoglucosidasius; B. thermolactis; B. thermoleovorans; B. thermophiles; B. thermoruber; B. thermosphaericus; B. thiaminolyticus; B. thioparans; B. thuringiensis; B. tianshenii; B. trypoxylicola; B. tusciae; B. validus; B. vallismortis; B. vedderi; B. velezensis; B. vietnamensis; B. vireti; B. vulcani; B. wakoensis; B. xiamenensis; B. xiaoxiensis; B. zanthoxyli; and B. zhanjiangensis.

With respect to the Genus Listeria, representative species include Listeria monocytogenes, Listeria seeligeri, Listeria ivanovii, Listeria welshimeri, Listeria marthii, Listeria innocua, Listeria grayi, Listeria fleischmannii, Listeria floridensis, Listeria aquatica, Listeria newyorkensis, Listeria cornellensis, Listeria rocourtiae, Listeria weihenstephanensis, Listeria grandensis, Listeria riparia, and Listeria booriae.

With respect to the Genus Staphylococcus, representative species include S. argenteus; S. arlettae; S. agnetis; S. aureus; S. auricularis; S. caeli; S. capitis; S. caprae; S. carnosus; S. caseolyticus; S. chromogenes; S. cohnii; S. cornubiensis; S. condiment; S. debuckii; S. delphini; S. devriesei; S. edaphicus; S. epidermidis; S. equorum; S. felis; S. fleurettii; S. gallinarum; S. haemolyticus; S. hominis; S. hyicus; S. intermedius; S. jettensis; S. kloosii; S. leei; S. lentus; S. lugdunensis; S. lutrae; S. lyticans; S. massiliensis; S. microti; S. muscae; S. nepalensis; S. pasteuri; S. petrasii; S. pettenkoferi; S. piscifermentans; S. pseudintermedius; S. pseudolugdunensis; S. pulvereri; S. rostri; S. saccharolyticus; S. saprophyticus; S. schleiferi; S. schweitzeri; S. sciuri; S. simiae; S. simulans; S. stepanovicii; S. succinus; S. vitulinus; S. warneri; and S. xylosus.

With respect to the Genus Varibaculum, representative species include V. anthropic; V. cambriense; V. massiliense; and V. timonense.

With respect to the Genus Murdochiella, representative species include Murdochella alacer; Murdochella Antarctica; Murdochella crispate; Murdochella levifoliata; Murdochella lobate; Murdochella macrina; Murdochella superlata (synonym of Papuliscala superlata); and Murdochella tertia.

With respect to the Genus Ruminiclostridium, representative species include Ruminiclostridium cellobioparum; Ruminiclostridium cellulolyticum; Ruminiclostridium hungatei; Ruminiclostridium josui; Ruminiclostridium papyrosolvens; Ruminiclostridium sufflavum; and Ruminiclostridium sp. MA18.

With respect to the Genus Prevotella, representative species include Prevotella albensis; Prevotella amnii; Prevotella bergensis; Prevotella bivia; Prevotella brevis; Prevotella bryantii; Prevotella buccae; Prevotella buccalis; Prevotella copri; Prevotella dentalis; Prevotella denticola; Prevotella disiens; Prevotella histicola; Prevotella intermedia; Prevotella maculosa; Prevotella marshii; Prevotella melaninogenica; Prevotella micans; Prevotella multiformis; Prevotella nigrescens; Prevotella oralis; Prevotella oris; Prevotella oulorum; and Prevotella pallens.

With respect to the Genus Ezakiella, representative species include Ezakiella coagulans; Ezakiella massiliensis; Ezakiella peruensis; and Ezakiella massiliensis.

With respect to the Genus Eisenbergiella, representative species include E. massiliensis; and E. tayi.

With respect to the Genus Anaerococcus, representative species include Anaerococcus hydrogenalis; Anaerococcus lactolyticus; Anaerococcus octavius; Anaerococcus prevotii; Anaerococcus tetradius; Anaerococcus vaginalis; Anaerococcus murdochii; Anaerococcus degenerii; Anaerococcus provencensis; Anaerococcus senegalensis; Anaerococcus rubiinfantis; Anaerococcus marasmi; Anaerococcus urinomassiliensis; and Anaerococcus nagyae.

With respect to the Genus Barnesiella, representative species include B. intestinihominis and B. viscericola.

With respect to the Family Bacteroidaceae, representative genera include Acetofilamentum; Acetomicrobium; Acetothermus; Anaerorhabdus; Bacteroides; Capsularis.

With respect to the Family Barnesiellaceae, representative genera include Barnesiella and Coprobacter.

With respect to the Genus Bacteroides, representative species include B. acidifaciens; B. barnesiaes; B. caccae; B. caecicola; B. caecigallinarum; B. cellulosilyticus; B. cellulosolvens; B. clarus; B. coagulans; B. coprocola; B. coprophilus; B. coprosuis; B. distasonis (which may be considered Parabacteroides distasonis); B. dorei; B. eggerthii; B. gracilis; B. faecichinchillae; B. faecis; B. finegoldii; B. fluxus; B. fragilis; B. galacturonicus; B. gallinaceum; B. gallinarum; B. goldsteinii; B. graminisolvens; B. helcogene; B. intestinalis; B. luti; B. massiliensis; B. melaninogenicus; B. nordii; B. oleiciplenus; B. oris; B. ovatus; B. paurosaccharolyticus; B. plebeius; B. polypragmatus; B. propionicifaciens; B. putredinis; B. pyogenes; B. reticulotermitis; B. rodentium; B. salanitronis; B. salyersiae; B. sartorii; B. sediment; B. stercoris; B. suis; B. tectus; B. thetaiotaomicronl B. uniformis; B. vulgatus; B. xylanisolvens; and B. xylanolyticus.

With respect to the Genus Fournierella, representative species include Fournierella massiliensis.

With respect to the family Porphyromonadaceae, representative Genera include Barnesiella; Candidatus Vestibaculum; Coprobacter; Dysgonomonas; Falsiporphyromonas; Fermentimonas; Gabonia; Gabonibacter; Lascolabacillus; Macellibacteroides; Microbacter; Muribaculum; Parabacteroides; Porphyromonas; Proteiniphilum; Sanguibacteroides; and Tannerella.

With respect to the Genus Peptoniphilus, representative species include P. asaccharolyticus; P. catoniae; P. coxii; P. duerdenii; P. gorbachii; P. harei; P. ivorii; P. koenoeneniae; P. lacrimalis; P. lacydonensis; P. methioninivorax; P. olsenii; P. senegalensis; P. stercorisuis; P. timonensis; P. tyrrelliae; and P. urinimassiliensis.

With respect to the Genus Porphyromonas, representative species include P. asaccharolytica; P. bennonis; P. cangingivalis; P. canoris; P. catoniae; P. circumdentaria; P. crevioricanis; P. endodontalis; P. gingivalis; P. gingivicanis; P. gulae; P. levii; P. macacae; P. pasteri; P. pogonae; P. somerae; and P. uenonis.

With respect to the Genus GCA-900066575, representative species include sp002160765; sp002160825; sp900066385; and sp900553635.

With respect to the Genus Lachnoclostridium, representative species include Lachnoclostridium pacaense; Lachnoclostridium touaregense; Lachnoclostridium bouchesdurhonense; Lachnoclostridium phytofermentans; Lachnoclostridium sp. YL32; and Lachnoclostridium massiliosenegalense.

With respect to the Genus Intestinimonas, representative species include Intestinimonas butyriciproducens and Intestinimonas massiliensis.

With respect to the Genus Flavonifractor, a representative species is Flavonifractor plautii.

4. Cytokine Release Syndrome Risk

In particular embodiments, the composition of the gut microbiome acts as a biomarker for whether or not an immunotherapy, such as cells expressing one or more engineered antigen receptors (including CAR T-cell therapy), will be toxic to the individual. In specific embodiments, the composition of the gut microbiome acts as a biomarker for the risk of the individual having cytokine release syndrome (CRS) as a result of the CAR T-cell therapy. The gut microbiome of the individual may allow for the determination of the grade of CRS, and such information may impact whether or not the individual will receive the CAR T-cell therapy, or whether or not adjustment to the treatment regimen is warranted.

In particular embodiments, the gut microbiome analysis for the individual determines that the individual will have a CRS grade of 0, 1, 2, 3, or 4.

In specific embodiments, when the individual has one or more of the following microbes, the individual will have a CRS grade of 2, 3, or 4: Species Lactobacillus rhamnosus; Species Parabacteroides goldsteinii; Species Olsenella uli; Species Fusobacterium varium; Genus Porphyrobacter; Species Dialister succinatiphilus; Species Faecalitalea cylindroides; Species Porphyrobacter sanguineus; Species Ruminiclostridium 9 unclass; Family Sphingomonadaceae; Order Sphingomonadales; and Genus Olsenella.

In specific embodiments, when an individual is determined to have a particular number of the aforementioned microbes, such as at least 10, 15, 20, 25, 30, 35, 40, 45, 50, 55, 60, 65, 70, 75, 80, 85, 90, 95, or greater than 95% of the aforementioned microbes, the individual will have a CRS grade of 2, 3, or 4 or has an increased chance of having a CRS grade of 2, 3, or 4 compared to an individual that does not have the corresponding microbe(s).

In such cases, the individual as a result of the analysis may or may not be provided a therapeutically effective amount of the CAR T-cell therapy. In specific aspects, the treatment regimen may be modified to further reduce the risk of CRS for the individual, such as modification of the CAR T-cells and/or dosage (such as lowering) thereof. For example, the target antigen of the CAR may be changed; one or more additional or different costimulatory domains may be utilized in the CAR; an additional CAR targeting an additional target antigen may be utilized in the cells; one or more cytokines may be avoided with the CAR T-cell therapy, and so forth. The individual may or may not be subject to an additional cancer therapy. The individual may be given one or more probiotic compositions and/or one or more fecal transplantations, including that comprise one or more microbes that over come any deficiencies in the individual's gut microbiome.

In other embodiments, the gut microbiome analysis for the individual determines that the individual will have a CRS grade of 0 or 1 or has a greater chance of having a CRS grade of 0 or 1 compared to an individual that does not have the corresponding microbe(s). In specific embodiments, when the individual has one or more of the following microbes, the individual will have (or is at risk of having, compared to an individual that lacks these microbes) a CRS grade of 0 or 1: Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species Clostridium celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; Species Veillonella parvula.

In specific embodiments, when an individual is determined to have a particular number of the aforementioned microbes, such as at least 10, 15, 20, 25, 30, 35, 40, 45, 50, 55, 60, 65, 70, 75, 80, 85, 90, 95, or greater than 95% of the aforementioned microbes, the individual will have a CRS grade of 0 or 1 or has an increased chance of having a CRS grade of 0 or 1 compared to an individual that does not have the corresponding microbe(s).

In such cases, the individual as a result of the analysis is able to be provided a therapeutically effective amount of the CAR T-cell therapy. In specific aspects, however, the treatment regimen may be modified to further reduce the risk of CRS for the individual, such as modification of the CAR T-cells and/or dosage (such as lowering) thereof. For example, the target antigen of the CAR may be changed; one or more additional or different costimulatory domains may be utilized in the CAR; an additional CAR targeting an additional target antigen may be utilized in the cells; one or more cytokines may be avoided with the CAR T-cell therapy, and so forth. The individual may or may not be subject to an additional cancer therapy. The individual may be given one or more probiotic compositions and/or one or more fecal transplantations, including that comprise one or more microbes that over come any deficiencies in the individual's gut microbiome.

Below, particular species are indicated from Order, Family, and Genera referred to in the Cytokine Release Syndrome Risk lists above.

With respect to the Genus Porphyrobacter, representative species include P. colymbi; P. cryptus; P. dokdonensis; P. donghaensis; P. neustonensis; P. sanguineus; and P. tepidarius.

With respect to the Family Sphingomonadaceae, representative Genera include Blastomonas; Citrimicrobium; Citromicrobium; Hankyongella; Hephaestia; Lutibacterium; Novosphingobium; Pacificimonas; Parablastomonas; Parasphingopyxis; Polymorphobacter; Rhizorhabdus; Rhizorhapis; Sandaracinobacter; Sandarakinorhabdus; Sphingoaurantiacus; Sphingobium; Sphingomicrobium; Sphingomonas; Sphingopyxis; Sphingorhabdus; Sphingosinicella; Stakelama; and Zymomonas.

With respect to the Order Sphingomonadales, representative Genera include Altererythrobacter; Blastomonas (a representative species is Blastomonas aquatic); Citrimicrobium; Citromicrobium; croceicoccus; Erythrobacter; Porphyrobacter; Sphingobium; Sphingomonadaceae; Sphingomonadales; Sphingomonas; Sphingopyxis; and Zymomonas (a representative species is Zymomonas mobilis).

With respect to Genus Altererythrobacter, representative species include Altererythrobacter aerius; Altererythrobacter aestiaquae; Altererythrobacter aestuarii; Altererythrobacter aquaemixtae; Altererythrobacter aquiaggeris; Altererythrobacter atlanticus; Altererythrobacter aurantiacus; Altererythrobacter buctensis; Altererythrobacter confluentis; Altererythrobacter deserti; Altererythrobacter dongtanensis; Altererythrobacter endophyticus; Altererythrobacter epoxidivorans; Altererythrobacter flavus; Altererythrobacter fulvus; Altererythrobacter gangjinensis; Altererythrobacter indicus; Altererythrobacter ishigakiensis; Altererythrobacter lauratis; Altererythrobacter luteolus; Altererythrobacter mangrove; Altererythrobacter marensis; Altererythrobacter marinus; Altererythrobacter namhicola; Altererythrobacter oceanensis; Altererythrobacter rigui; Altererythrobacter salegens; Altererythrobacter sediminis; Altererythrobacter soli; Altererythrobacter troitsensis; Altererythrobacter xiamenensis; Altererythrobacter xinjiangensis; and Altererythrobacter xixiisoli.

With respect to Genus Erythrobacter, representative species include Erythrobacter aquimaris; Erythrobacter aquimixticola; Erythrobacter arachoides; Erythrobacter atlanticus; Erythrobacter citreus; Erythrobacter flavus; Erythrobacter gaetbuli; Erythrobacter gangjinensis; Erythrobacter jejuensis; Erythrobacter litoralis; Erythrobacter longus; Erythrobacter luteus; Erythrobacter lutimaris; Erythrobacter marinus; Erythrobacter nanhaisediminis; Erythrobacter odishensis; Erythrobacter pelagi; Erythrobacter seohaensis; Erythrobacter vulgaris; Erythrobacter xanthus; Erythrobacteraceae; Erythromicrobium; Erythromicrobium ramosum; and Erythrobacter aquimaris.

With respect to Genus Novosphingobium, representative species include Novosphingobium acidiphilum; Novosphingobium aquaticum; Novosphingobium aquiterrae; Novosphingobium arabidopsis; Novosphingobium barchaimii; Novosphingobium chloroacetimidivorans; Novosphingobium endophyticum; Novosphingobium fluoreni; Novosphingobium fuchskuhlense; Novosphingobium gossypii; Novosphingobium hassiacum; Novosphingobium indicum; Novosphingobium kunmingense; Novosphingobium lentum; Novosphingobium lindaniclasticum; Novosphingobium malaysiense; Novosphingobium marinum; Novosphingobium mathurense; Novosphingobium naphthalenivorans; Novosphingobium nitrogenifigens; Novosphingobium oryzae; Novosphingobium panipatense; Novosphingobium rhizosphaerae; Novosphingobium sediminicola; Novosphingobium soli; Novosphingobium taihuense; and Novosphingobium tardaugens.

With respect to Genus Porphyrobacter, representative species include Porphyrobacter colymbi; Porphyrobacter cryptus; Porphyrobacter dokdonensis; Porphyrobacter donghaensis; Porphyrobacter neustonensis; and Porphyrobacter tepidarius.

With respect to Genus Sphingobium, representative species include Sphingobium chlorophenolicum; Sphingobium francense; Sphingobium indicum; and Sphingobium japonicum.

With respect to Genus Sphingopyxis, representative species include Sphingopyxis alaskensis; Sphingopyxis baekryungensis; Sphingopyxis bauzanensis; Sphingopyxis chilensis; Sphingopyxis flava; Sphingopyxis flavimaris; Sphingopyxis fribergensis; Sphingopyxis ginsengisoli; Sphingopyxis granuli; Sphingopyxis indica; Sphingopyxis italic; Sphingopyxis nepalensis; Sphingopyxis panaciterrae; Sphingopyxis panaciterrulae; Sphingopyxis soli; Sphingopyxis solisilvae; Sphingopyxis taejonensis; Sphingopyxis ummariensis; Sphingopyxis witflariensis; and Sphingosinicella humi.

With respect to the Genus Olsenella, representative species include O. profuse; O. scatoligenes; O. uli; and O. umbonata.

With respect to the Family Eubacteriaceae, representative Genera include Acetobacterium; Alkalibacter; Alkalibaculum; Aminicella; Anaerofustis; Eubacterium; Garciella; Intestinibacillus; Irregularibacter; Pseudoramibacter; and Rhabdanaerobium.

With respect to Genus Acetobacterium, representative species include Acetobacterium bakii; Acetobacterium carbinolicum; Acetobacterium fimetarium; Acetobacterium malicum; Acetobacterium paludpsum; Acetobacterium tundra; Acetobacterium wieringae; and Acetobacterium woodii.

With respect to Genus Alkalibacter, a representative species is A. saccharofermentans.

With respect to the Genus Alkalibaculum, a representative species is A. bacchi.

With respect to the Genus Aminicella, a representative species is Aminicella lysinilytica.

With respect to the Genus Anaerofustis, a representative species is A. stercorihominis.

With respect to the Genus Eubacterium, representative species include Eubacterium aggregans; Eubacterium angustum; Eubacterium barkeri; Eubacterium brachy; Eubacterium budayi; Eubacterium callanderi; Eubacterium cellulosolvens; Eubacterium combesii; Eubacterium coprostanoligenes; Eubacterium dolichum; Eubacterium eligens; Eubacterium hallii; Eubacterium infirmum; Eubacterium limosum; Eubacterium minutum; Eubacterium multiforme; Eubacterium nitritogenes; Eubacterium nodatum; Eubacterium oxidoreducens; Eubacterium plexicaudatum; Eubacterium pyruvativorans; Eubacterium ramulus; Eubacterium rectale; Eubacterium ruminantium; Eubacterium saphenum; Eubacterium siraeum; Eubacterium sulci; Eubacterium tarantellae; Eubacterium tenue; Eubacterium tortuosum; Eubacterium uniforme; Eubacterium ventriosum; Eubacterium xylanophilum; and Eubacterium yurii.

With respect to the Genus Garciella, a representative species is G. nitratireducens.

With respect to the Genus Intestinibacillus, a representative species is Intestinibacillus massiliensis.

With respect to the Genus Irregularibacter, a representative species is Irregularibacter muris.

With respect to the Genus Pseudoramibacter, a representative species is P. alactolyticus.

With respect to the Genus Rhabdanaerobium, a representative species is Rhabdanaerobium thermarum.

With respect to the Genus Ruminococcus, representative species include Ruminococcus albus; Ruminococcus bromii; Ruminococcus callidus; Ruminococcus flavefaciens; Ruminococcus gauvreauii; Ruminococcus gnavus; Ruminococcus lactaris; Ruminococcus obeum; and Ruminococcus torques.

With respect to the Genus Turicibacter, a representative species is Turicibacter sanguinis.

With respect to the Genus Veillonella, representative species include V. tobetsuensis; V. magna; V. criceti; V. ratti; V. montpellierensis; V. caviae; V. dispar; V. parvula; V. rogosae; V. atypica; V. denticariosi; and V. rodentium.

With respect to the Genus Atopobium, representative species include A. deltae; A. fossor; A. minutum; A. parvulum; A. rimae; and A. vaginae.

With respect to the Genus Lactonifactor, a representative species is Lactonifactor longoviformis.

With respect to the Family Peptostreptococcaceae, representative Genera include Acetoanaerobium; Asaccharospora; Clostridioides; Criibacterium; Filifactor; Intestinibacter; Paeniclostridium; Paraclostridium; Peptoanaerobacter; Peptoclostridium; Peptostreptococcus; Proteocatella; Romboutsia; Sporacetigenium; Tepidibacter; and Terrisporobacter.

With respect to the Genus Acetoanaerobium, a representative species is A. noterae.

With respect to the Genus Asaccharospora, a representative species is Asaccharospora irregularis.

With respect to the Genus Clostridioides, representative species include Clostridioides difficile and Clostridioides mangenotii.

With respect to the Genus Criibacterium, a representative species is Criibacterium bergeronii.

With respect to the Genus Filifactor, representative species include Filifactor villosus and Filifactor alocis.

With respect to the Genus Intestinibacter, a representative species is Intestinibacter bartlettii.

With respect to the Genus Paeniclostridium, representative species include Paeniclostridium sordellii and Paeniclostridium ghonii.

With respect to the Genus Paraclostridium, representative species include Paraclostridium bifermentans and Paraclostridium benzoelyticum.

With respect to the Genus Peptoanaerobacter, a representative species is Peptoanaerobacter stomatis.

With respect to the Genus Peptoclostridium, a representative species is Peptoclostridium difficile.

With respect to the Genus Peptostreptococcus, representative species include Peptostreptococcus anaerobius; Peptostreptococcus asaccharolyticus; Peptostreptococcus canis; Peptostreptococcus harei; Peptostreptococcus hydrogenalis; Peptostreptococcus indoliticus; Peptostreptococcus ivorii; Peptostreptococcus lacrimalis; Peptostreptococcus lactolyticus; Peptostreptococcus magnus; Peptostreptococcus micros; Peptostreptococcus octavius; Peptostreptococcus prevotii; Peptostreptococcus tetradius; Peptostreptococcus russellii; Peptostreptococcus stomatis; and Peptostreptococcus vaginalis.

With respect to the Genus Proteocatella, a representative species is Proteocatella sphenisci.

With respect to the Genus Romboutsia, representative species include Romboutsia timonensis; Romboutsia hominis; Romboutsia lituseburensis; and Romboutsia ilealis.

With respect to the Genus Sporacetigenium, representative species include S. poracetigenium and Sporacetigenium mesophilum.

With respect to the Genus Tepidibacter, representative species include T. formicigenes; T. mesophilus; and T. thalassicus.

With respect to the Genus Terrisporobacter, representative species include Terrisporobacter glycolicus; Terrisporobacter mayombei; and Terrisporobacter petrolearius.

5. Immune Effector Cell-Associated Neurotoxicity Syndrome Risk

In particular embodiments, the gut microbiome of an individual in need of immunotherapy such as CAR T-cell therapy is analyzed prior to the therapy to determine the risk of the individual of having immune effector cell-associated neurotoxicity syndrome (ICANS). In particular embodiments, the gut microbiome of an individual is analyzed prior to CAR T-cell therapy to determine the grade (0, 1, 2, 3, or 4) of ICANS that the individual is susceptible to, compared to individuals that do not have the same microbe(s).

In specific embodiments, an individual is at risk of having ICANS grade of 3 or 4 if the gut microbiome has one or more of the following microbes: Species [Clostridium] lavalense; Genus Cuneatibacter; Family Clostridiales vadin BB60 group; Genus Clostridiales vadin BB60 group; Species Clostridiales vadin BB60 group ge unclass; Species [Clostridium] hylemonae; Species Anaerotruncus rubiinfantis; Genus Ruminococcaceae UCG 004; Species Eisenbergiella massiliensis; Genus Hydrogenoanaerobacterium; Genus Ruminococcaceae UCG 008; Species Intestinibacillus massiliensis; Genus Raoultibacter; Species Monoglobus pectinilyticus; Family Bacillaceae; Species Pseudomonas aeruginosa; Species Bacillus hisashii; Species Caecibacter massiliensis; and Species Prevotella multisaccharivorax. In particular embodiments, the individual is at risk of having ICANS grade of 3 or 4 if the gut microbiome has 1, 2, 3, 4, 5, 6, 7, 8, 9, 10, 11, 12, 13, 14, 15, 16, 17, 18, 19, or 20 or more of the microbes. In at least some cases, the individual is at risk of having ICANS grade of 3 or 4 if the gut microbiome has at least 10, 15, 20, 25, 30, 35, 40, 45, 50, 55, 60, 65, 70, 75, 80, 85, 90, 95, or greater than 95% of the aforementioned microbes.

In cases where it is determined based on the gut microbiome that the individual will have an ICANS grade of 3 or 4, the individual may or may not be given the CAR T-cell therapy. In some cases when the individual will still receive a CAR T-cell therapy despite having one or more of the aforementioned microbes, the CAR T-cell therapy may be given in a different (such as lower) dosage and/or may be modified to be able to overcome the risk of the individual having ICANS. For example, the target antigen of the CAR may be changed; additional or different costimulatory domains may be utilized in the CAR; an additional CAR targeting an additional target antigen may be utilized in the cells; one or more cytokines may be avoided with the CAR T-cell therapy, and so forth.

In some embodiments, the gut microbiome of an individual is analyzed and it is determined that the individual will have or is at risk of having an ICANS grade of 0, 1, or 2, including at risk compared to an individual that does not have the corresponding one or more microbes. In specific embodiments, the gut microbiome of the individual has one or more of the following: Genus Corynebacterium; Species Lactobacillus sakei; Species Veillonella dispar; Species Streptococcus salivarius; Species Coprococcus eutactus. In some cases, the gut microbiome has the majority or all of the aforementioned list. The individual may have 1, 2, 3, 4, or 5 microbes from the aforementioned list.

In particular embodiments, the individual following this determination is given a therapeutically effective amount of the CAR T-cell therapy. In some cases, the CAR T-cell therapy in an effort to further reduce the risk of ICANS may be given in a different (such as lower) dosage and/or may be modified to be able to overcome the risk of the individual having ICANS. For example, the target antigen of the CAR may be changed; additional or different costimulatory domains may be utilized in the CAR; an additional CAR targeting an additional target antigen may be utilized in the cells; one or more cytokines may be avoided with the CAR T-cell therapy, and so forth. The individual may be given one or more probiotic compositions and or one or more fecal transplantations, including that comprise one or more microbes that over come any deficiencies in the individual's gut microbiome.

Below, particular species are indicated from Order, Family, and Genera referred to in the ICANS Risk list above.

With respect to the Genus Cuneatibacter, a representative species is C. caecimuris.

With respect to the Genus Hydrogenoanaerobacterium, a representative species is Hydrogenoanaerobacterium saccharovorans.

With respect to the Genus Raoultibacter, representative species include Raoultibacter timonensis and Raoultibacter massiliensis.

With respect to the Family Bacillaceae, representative Genera include Aeribacillus; Aliibacillus; Alkalibacillus; Alkalicoccus; Alkalilactibacillus; Allobacillus; Alteribacillus; Amphibacillus; Amylobacillus; Anaerobacillus; Anoxybacillus; Aquibacillus; Aquisalibacillus; Aureibacillus; Bacillus; Caldalkalibacillus; Caldibacillus; Calditerricola; Cerasibacillus; Compostibacillus; Desertibacillus; Domibacillus; Edaphobacillus; Falsibacillus; Fermentibacillus; Fictibacillus; Filobacillus; Geobacillus; Geomicrobium; Gracilibacillus; Halalkalibacillus; Halobacillus; Halolactibacillus; Hydrogenibacillus; Lentibacillus; Lysinibacillus; Marinococcus; Massilibacterium; Melghiribacillus; Microaerobacter; Natribacillus; Natronobacillus; Numidum; Oceanobacillus; Ornithinibacillus; Parageobacillus; Paraliobacillus; Paralkalibacillus; Paucisalibacillus; Pelagirhabdus; Piscibacillus; Polygonibacillus; Pontibacillus; Pradoshia; Pseudobacillus; Pseudogracilibacillus; Psychrobacillus; Pueribacillus; Quasibacillus; Rubeoparvulum; Saccharococcus; Salibacterium; Salimicrobium; Salinibacillus; Salipaludibacillus; Salirhabdus; Salisediminibacterium; Saliterribacillus; Salsuginibacillus; Sediminibacillus; Sinibacillus; Streptohalobacillus; Swionibacillus; Tenuibacillus; Tepidibacillus; Terribacillus; Terrilactibacillus; Texcoconibacillus; Thalassobacillus; Thalassorhabdus; Thermolongibacillus; Virgibacillus; and Vulcanibacillus.

With respect to the Genus Aeribacillus, a representative species is A. pallidus.

With respect to the Genus Aliibacillus, a representative species is Aliibacillus thermotolerans.

With respect to the Genus Alkalibacillus, representative species include A. almallahensis; A. filiformis; A. flavidus; A. haloalkaliphilus; A. halophilus; A. salilacus; and A. silvisoli.

With respect to the Genus Alkalilactibacillus, a representative species is Alkalilactibacillus ikkensis.

With respect to the Genus Alkalicoccus, representative species include Alkalicoccus saliphilus and Alkalicoccus halolimnae.

With respect to the Genus Allobacillus, a representative species is A. halotolerans.

With respect to the Genus Alteribacillus, representative species include A. alkaliphilus; A. bidgolensis; A. iranensis; and A. persepolensis.

With respect to the Genus Amphibacillus, representative species include A. cookii; A. fermentum; A. iburiensis; A. indicireducens; A. jilinensis; A. marinus; A. sediminis; A. tropicus; and A. xylanus.

With respect to the Genus Amylobacillus, a representative species is A. thermophiles.

With respect to the Genus Anaerobacillus, representative species include A. alkalidiazotrophicus; A. alkalilacustris; and A. arseniciselenatis.

With respect to the Genus Anoxybacillus, A. amylolyticus; A. ayderensis; A. bogrovensis; A. caldiproteolyticus; A. calidus; A. contaminans; A. eryuanensis; A. flavithermus; A. gonensis; A. kamchatkensis; A. kaynarcensis; A. kestanbolensis; A. mongoliensis; A. pushchinoensis; A. rupiensis; A. salavatliensis; A. tengchongensis; A. tepidamans; A. thermarum; A. vitaminiphilus; and A. voinovskiensis.

With respect to the Genus Aquibacillus, representative species include A. albus; A. halophilus; A. koreensis; and A. salifodinae.

With respect to the Genus Aquisalibacillus, a representative species is A. elongates.

With respect to the Genus Aureibacillus, a representative species is Aureibacillus halotolerans.

With respect to the Genus Caldalkalibacillus, representative species include C. thermarum and C. uzonensis.

With respect to the Genus Caldibacillus, a representative species is C. debilis.

With respect to the Genus Calditerricola, representative species include C. satsumensis and C. yamamurae.

With respect to the Genus Cerasibacillus, a representative species is C. quisquiliarum.

With respect to the Genus Compostibacillus, a representative species is C. humi.

With respect to the Genus Desertibacillus, a representative species is Desertibacillus haloalkaliphilus.

With respect to the Genus Domibacillus, representative species include D. antri; D. enclensis; D. indicus; D. iocasae; D. robiginosus; and D. tundra.

With respect to the Genus Edaphobacillus, a representative species is E. lindanitolerans.

With respect to the Genus Falsibacillus, a representative species is F. pallidus.

With respect to the Genus Fermentibacillus, a representative species is F. polygoni.

With respect to the Genus Fictibacillus, representative species include F. arsenicus; F. barbaricus; F. enclensis; F. gelatini; F. halophilus; F. acauensis; F. nanhaiensis; F. phosphorivorans; F. rigui; and F. solisalsi.

With respect to the Genus Filobacillus, a representative species is F. milensis.

With respect to the Genus Geobacillus, representative species include G. caldoxylosilyticus; G. galactosidasius; G. icigianus; G. jurassicus; G. kaustophilus; G. lituanicus; G. stearothermophilus; G. subterraneus; G. thermantarcticus; G. thermocatenulatus; G. thermodenitrificans; G. thermoglucosidasius; G. thermoleovorans; G. toebii; G. uzenensis; and G. vulcani.

With respect to the Genus Geomicrobium, representative species include Geomicrobium halophilum and Geomicrobium sediminis.

With respect to the Genus Gracilibacillus, representative species include G. alcaliphilus; G. boraciitolerans; G. bigeumensis; G. dipsosauri; G. halophilus; G. halotolerans; G. kekensis; G. lacisalsi; G. massiliensis; G. orientalis; G. quinghaiensis; G. saliphilus; G. thailandensis; and G. ureilyticus.

With respect to the Genus Halalkalibacillus, a representative species is H. halophilus.

With respect to the Genus Halobacillus, representative species include H. aidingensis; H. alkaliphilus; H. andaensis; H. campisalis; H. dabanensis; H. faecis; H. halophilus; H. karajensis; H. kuroshimensis; H. litoralis; H. locisalis; H. mangrove; H. naozhouensis; H. profundi; H. salinus; H. salsuginis; H. seohaensis; H. trueperi; and H. yeomjeoni.

With respect to the Genus Halolactibacillus, representative species include H. alkaliphilus; H. halophilus; and H. miurensis.

With respect to the Genus Hydrogenibacillus, a representative species is H. schlegelii.

With respect to the Genus Lentibacillus, representative species include L. garicola; L. halodurans; L. halophilus; L. jeotgali; L. juripiscarius; L. kapialis; L. lacisalsi; L. persicus; L. salaries; L. salicampi; L. salinarum; and L. salis.

With respect to the Genus Lysinibacillus, representative species include L. sphaericus and L. fusiformis.

With respect to the Genus Marinococcus, representative species include M. halophilus; M. halotolerans; M. luteus; M. salis; and M. tarijensis.

With respect to the Genus Massilibacterium, a representative species is Massilibacterium senegalense.

With respect to the Genus Melghiribacillus, a representative species is M. thermohalophilus.

With respect to the Genus Microaerobacter, a representative species is M. geothermalis.

With respect to the Genus Natribacillus, a representative species is N. halophilus.

With respect to the Genus Natronobacillus, a representative species is N. azotifigens.

With respect to the Genus Numidum, a representative species is Numidum massiliense.

With respect to the Genus Oceanobacillus, O. arenosus; O. bengalensis; O. caeni; O. chironomi; O. chungangensis; O. damuensis; O. iheyensis; O. indicireducens; O. kapialis; O. kimchi; O. limi; O. locisalsi; O. luteolus; O. neutriphilus; O. oncorhynchi; O. pacificus; O. picturae; O. polygoni; O. profundus; O. rekensis; and O. sojae.

With respect to the Genus Ornithinibacillus, representative species include O. bavariensis; O. californiensis; O. contaminans; O. halophilus; O. heyuanensis; and O. scapharcae.

With respect to the Genus Parageobacillus, representative species include Parageobacillus caldoxylosilyticus; Parageobacillus genomospecies 1; Parageobacillus thermantarcticus; Parageobacillus thermoglucosidasius; and Parageobacillus toebii.

With respect to the Genus Paraliobacillus, representative species include P. quinghaiensis; P. ryukyuensis; and P. sediminis.

With respect to the Genus Paralkalibacillus, a representative species is Paralkalibacillus indicireducens.

With respect to the Genus Paucisalibacillus, representative species include P. algeriensis and P. globulus.

With respect to the Genus Pelagirhabdus, representative species include P. alkalitolerans and P. fermentum.

With respect to the Genus Piscibacillus, representative species include P. halophilus and P. salipiscarius.

With respect to the Genus Polygonibacillus, a representative species is P. indicireducens.

With respect to the Genus Pontibacillus, representative species include P. chungwhensis; P. halophilus; P. litoralis; P. marinus; P. salicampi; P. salipaludis; and P. yanchengensis.

With respect to the Genus Pradoshia, a representative species is Pradoshia eiseniae.

With respect to the Genus Pseudobacillus, a representative species is Pseudobacillus badius.

With respect to the Genus Pseudogracilibacillus, representative species include P. marinus; P. endophyticus; and P. auburnensis.

With respect to the Genus Psychrobacillus, representative species include P. insolitus; P. psychrodurans; P. psychrotolerans; and P. soli.

With respect to the Genus Pueribacillus, a representative species is Pueribacillus theae.

With respect to the Genus Quasibacillus, a representative species is Quasibacillus thermotolerans.

With respect to the Genus Rubeoparvulum, a representative species is Rubeoparvulum massiliense.

With respect to the Genus Saccharococcus, a representative species is S. thermophiles.

With respect to the Genus Salibacterium, representative species include S. halochares; S. halotolerans; S. qingdaonense; and S. lacus.

With respect to the Genus Salimicrobium, representative species include S. album; S. flavidum; S. halophilum; S. jeotgali; S. luteum; and S. salexigens.

With respect to the Genus Salinibacillus, representative species include S. aidingensis; S. kushneri; and S. xinjiangensis.

With respect to the Genus Salipaludibacillus, representative species include S. agaradhaerens; S. aurantiacus; and S. neizhouensis.

With respect to the Genus Salirhabdus, representative species include S. euzebyi and S. salicampi.

With respect to the Genus Salisediminibacterium, representative species include S. haloalkalitolerans; S. halotolerans; and S. locisalis.

With respect to the Genus Saliterribacillus, a representative species is S. persicus.

With respect to the Genus Salsuginibacillus, representative species include S. halophilus and S. kocurii.

With respect to the Genus Sediminibacillus, representative species include S. albus; S. halophilus; and S. massiliensis.

With respect to the Genus Sinibacillus, a representative species is S. soli.

With respect to the Genus Streptohalobacillus, a representative species is S. salinus.

With respect to the Genus Swionibacillus, a representative species is Swionibacillus sediminis.

With respect to the Genus Tenuibacillus, representative species include Tenuibacillus multivorans and Tenuibacillus halotolerans.

With respect to the Genus Tepidibacillus, representative species include T. decaturensis; T. fermentans; and T. infernus.

With respect to the Genus Terribacillus, representative species include T. aidingensis; T. goriensis; T. halophilus; and T. saccharophilus.

With respect to the Genus Terrilactibacillus, a representative species is T. laevilacticus.

With respect to the Genus Texcoconibacillus, a representative species is T. texcoconensis.

With respect to the Genus Thalassobacillus, representative species include T. cyri; T. devorans; T. hwangdonensis; and T. pellis.

With respect to the Genus Thalassorhabdus, a representative species is Thalassorhabdus alkalitolerans.

With respect to the Genus Thermolongibacillus, representative species include T. altinsuensis and T. kozakliensis.

With respect to the Genus Virgibacillus, V. alimentarius; V. arcticus; V. byunsanensis; V. campisalis; V. carmonensis; V. chiguensis; V. dokdonensis; V. flavescens; V. halodenitrificans; V. halophilus; V. halotolerans; V. indicus; V. kapii; V. kekensis; V. litoralis; V. marismortui; V. natechei; V. necropolis; V. oceani; V. olivae; V. pantothenticus; V. phasianinus; V. picturae; V. profundi; V. proomii; V. salaries; V. salexigens; V. salinus; V. sediminis; V. siamensis; V. soli; V. subterraneus; and V. xinjiangensis.

With respect to the Genus Vulcanibacillus, a representative species is V. modesticaldus.

With respect to the Genus Corynebacterium, representative species include Corynebacterium accolens; Corynebacterium acetoacidophilum; Corynebacterium afermentans; Corynebacterium alimapuense; Corynebacterium alkanolyticum; Corynebacterium ammoniagenes; Corynebacterium amycolatum; Corynebacterium appendicis; Corynebacterium aquatimens; Corynebacterium aquilae; Corynebacterium argentoratense; Corynebacterium atrinae; Corynebacterium atypicum; Corynebacterium aurimucosum; Corynebacterium auris; Corynebacterium auriscanis; Corynebacterium bouchesdurhonense; Corynebacterium bovis; Corynebacterium callunae; Corynebacterium camporealensis; Corynebacterium canis; Corynebacterium capitovis; Corynebacterium casei; Corynebacterium caspium; Corynebacterium cervicis; Corynebacterium choanis; Corynebacterium ciconiae; Corynebacterium confusum; Corynebacterium coyleae; Corynebacterium crenatum; Corynebacterium crudilactis; Corynebacterium cyclohexanicum; Corynebacterium cystitidis; Corynebacterium defluvii; Corynebacterium deserti; Corynebacterium diphtheria; Corynebacterium doosanense; Corynebacterium durum; Corynebacterium efficiens; Corynebacterium epidermidicanis; Corynebacterium faecale; Corynebacterium falsenii; Corynebacterium fastidiosum; Corynebacterium felinum; Corynebacterium flavescens; Corynebacterium fournierii; Corynebacterium frankenforstense; Corynebacterium freiburgense; Corynebacterium freneyi; Corynebacterium genitalium; Corynebacterium geronticis; Corynebacterium glaucum; Corynebacterium glucuronolyticum; Corynebacterium glutamicum; Corynebacterium glyciniphilum; Corynebacterium godavarianum; Corynebacterium gottingense; Corynebacterium guangdongense; Corynebacterium hadale; Corynebacterium halotolerans; Corynebacterium hansenii; Corynebacterium heidelbergense; Corynebacterium humireducens; Corynebacterium ihumii; Corynebacterium imitans; Corynebacterium jeddahense; Corynebacterium jeikeium; Corynebacterium kefirresidentii; Corynebacterium kroppenstedtii; Corynebacterium kutscheri; Corynebacterium lactis; Corynebacterium lipophiloflavum; Corynebacterium lowii; Corynebacterium lubricantis; Corynebacterium macginleyi; Corynebacterium marinum; Corynebacterium maris; Corynebacterium massiliense; Corynebacterium mastitidis; Corynebacterium matruchotii; Corynebacterium melassecola; Corynebacterium minutissimum; Corynebacterium mucifaciens; Corynebacterium mustelae; Corynebacterium mycetoides; Corynebacterium nasicanis; Corynebacterium nephridii; Corynebacterium nuruki; Corynebacterium oculi; Corynebacterium otitidis; Corynebacterium pekinense; Corynebacterium pelargi; Corynebacterium phocae; Corynebacterium phoceense; Corynebacterium pilbarense; Corynebacterium pilosum; Corynebacterium pollutisoli; Corynebacterium propinquum; Corynebacterium provencense; Corynebacterium pseudodiphtheriticum; Corynebacterium pseudogenitalium; Corynebacterium pseudopelargi; Corynebacterium pseudotuberculosis; Corynebacterium pyruviciproducens; Corynebacterium renale; Corynebacterium resistens; Corynebacterium riegelii; Corynebacterium sanguinis; Corynebacterium segmentosum; Corynebacterium simulans; Corynebacterium singular; Corynebacterium sphenisci; Corynebacterium spheniscorum; Corynebacterium sputi; Corynebacterium stationis; Corynebacterium striatum; Corynebacterium suicordis; Corynebacterium sundsvallense; Corynebacterium tapiri; Corynebacterium terpenotabidum; Corynebacterium testudinoris; Corynebacterium thermoaminogenes; Corynebacterium thomssenii; Corynebacterium timonense; Corynebacterium tracheae; Corynebacterium tuberculostearicum; Corynebacterium tuscaniense; Corynebacterium ulcerans; Corynebacterium ulceribovis; Corynebacterium urealyticum; Corynebacterium ureicelerivorans; Corynebacterium urinapleomorphum; Corynebacterium uropygiale; Corynebacterium uterequi; Corynebacterium variabile; Corynebacterium vitaeruminis; and Corynebacterium xerosis.

### B. Examples of Compositions of the Disclosure

1. Fecal Transplant and Compositions

Embodiments of the disclosure encompass fecal transplants for individuals in need of adoptive cell therapy but that have an unsuitable gut microbiome that is at risk for lack of efficacy and/or that is at risk for toxicity for the individual.

In specific embodiments, the gut microbiome of an individual that is not the individual in need of CAR T-cell therapy is a donor for a fecal transplantation into the individual in need of the CAR T-cell therapy. In such cases, the donor individual not in need of CAR T-cell therapy is screened, and it is determined that their gut microbiome has a suitable composition based on parameters encompassed herein. That is, although the individual himself is not in need of the adoptive cell therapy, that individual's gut microbiome may be of a composition that (based on the parameters encompassed herein), would be suitable to be a donor. An effective amount of the feces from this individual may be transplanted into an individual that is in need of the CAR T-cell therapy. In specific embodiments, the feces of this individual (or combinations of other suitable donor individuals) may be stored in an off-the-shelf manner for later use by an individual in need thereof. In other cases it is not stored prior to use.

Fecal transplantation may be performed by colonoscopy or by nasoduodenal tube. During colonoscopy, a colonoscope is advanced through the entire colon. As the colonoscope is withdrawn, a fecal transplant composition is delivered through the colonoscopy into the colon. Methods of preparation of fecal transplantations are known in the art, such as U.S. Pat. No. 10,736,849.

In particular embodiments, one or more customized fecal transplant compositions are encompassed herein. The composition of the one or more fecal transplant compositions may or may not be tailored to address any deficiency in an individual's gut microbiome or to enhance an individual's gut microbiome. In some cases, the fecal transplant is considered to be off-the-shelf and comprises a standard one or more microbes to enhance immunotherapy of any kind, including CAR T-cell therapy. Such a fecal transplant may be given to an individual without having prior analysis of their gut microbiome. In certain embodiments, the fecal transplant composition is tailored to the specific deficiencies of the gut microbiome of the individual. In some cases, such a customized fecal transplant may or may not comprise all of the microbes that are considered to be deficient in the individual.

2. Probiotic Compositions

In particular embodiments, one or more probiotics compositions are encompassed herein. The composition of the one or more probiotic compositions may or may not be tailored to address any deficiency in an individual's gut microbiome or to enhance an individual's gut microbiome. In some cases, the probiotic is considered to be off-the-shelf and comprises a standard one or more microbes to enhance immunotherapy of any kind, including CAR T-cell therapy. Such a probiotic may be given to an individual without having prior analysis of their gut microbiome. The probiotic may comprise any one or more microbes listed herein as being associated with efficacious CAR T-cell therapy and/or not being associated with toxicity.

In certain embodiments, the probiotic composition is tailored to the specific deficiencies of the gut microbiome of the individual. In some cases, such a customized probiotic may or may not comprise all of the microbes that are considered to be deficient in the individual.

The individual may be given one or more probiotic compositions, including that comprise one or more microbes that overcome any deficiencies in the individual's gut microbiome. The probiotic may be given to enhance a CAR T-cell therapy and/or to reduce toxicity of the CAR T-cell therapy for the individual.

In specific embodiments, the probiotic comprises live microorganisms, which, when administered in adequate amounts, may enhance a CAR T-cell therapy and/or reduce toxicity of the CAR T-cell therapy for an individual. The probiotics may be available in foods and dietary supplements (for example, but not limited to capsules, tablets, and powders). Non-limiting examples of foods containing probiotics include dairy products such as yogurt, fermented and unfermented milk, smoothies, butter, cream, hummus, kombucha, salad dressing, miso, tempeh, nutrition bars, and some juices and soy beverages.

In cases wherein more than one microbe is in the probiotic, the ratio of the more than one microbe may or may not be substantially the same. For example, in some cases, two particular microbes in the composition may be at a ratio of 1:1, 1:2, 1:5, 1:10, 1:20, 1:50, 1:100, and so forth. In one embodiment, the probiotic composition comprises bacteria from at least two different bacterial species disclosed herein. Within a given composition, different bacterial strains can be contained in equal amounts (even combination) or in various proportions (uneven combinations) needed for achieving the maximal biological activity. For example, in a bacterial composition with two bacterial strains, the strains may be present in from a 1:10,000 ratio to a 1:1 ratio, from a 1:10,000 ratio to a 1:1,000 ratio, from a 1:1,000 ratio to a 1:100 ratio, from a 1:100 ratio to a 1:50 ratio, from a 1:50 ratio to a 1:20 ratio, from a 1:20 ratio to a 1:10 ratio, from a 1:10 ratio to a 1:1 ratio. For bacterial compositions comprising at least three bacterial strains, the ratio of strains may be chosen pairwise from ratios for bacterial compositions with two strains. For example, in a bacterial composition comprising bacterial strains A, B, and C, at least one of the ratios between strain A and B, the ratio between strain B and C, and the ratio between strain A and C may be chosen, independently, from the pairwise combinations above. In one specific embodiment, the invention encompasses administering two or more bacteria-containing compositions to the same subject. Such compositions can be administered simultaneously or sequentially.

The probiotic compositions of the disclosure can comprise, without limitation, e.g., live bacterial cells, conditionally lethal bacterial cells, inactivated bacterial cells, killed bacterial cells, spores (e.g., germination-competent spores), recombinant carrier strains, cell extract, and bacterially-derived products (natural or synthetic bacterially-derived products such as, e.g., bacterial antigens or bacterial metabolic products). In one specific embodiment, the probiotic composition comprises an excipient or a carrier that optimizes the seeding of one or more bacterial strains contained in the probiotic composition.

In one embodiment of any of the methods involving administration of a probiotic composition, the probiotic composition is reconstituted from a lyophilized preparation. In one embodiment of any of the methods involving administration of a probiotic composition, said probiotic composition comprises a buffering agent to adjust pH to a suitable number, such as 7.0.

Bacterial strains administered in probiotic compositions according to the methods of the present disclosure can comprise live bacteria. One or several different bacterial inoculants can be administered simultaneously or sequentially (including administering at different times). Such bacteria can be isolated from gastrointestinal (GI) microbiota and grown in culture. The present disclosure also comprises administering “bacterial analogues”, such as recombinant carrier strains expressing one or more heterologous genes derived from the relevant bacterial species. The use of such recombinant bacteria may allow the use of lower therapeutic amounts due to higher protein expression.

In one embodiment of any of the above methods involving administration of a probiotic composition, the probiotic composition comprises (i) a carrier and/or excipient and/or (ii) one or more prebiotic agents that stimulate growth and/or activity of one or more bacteria present in the composition. In one specific embodiment, the probiotic composition comprises an excipient or a carrier that optimizes the seeding of one or more bacterial strains contained in the probiotic composition.

In one embodiment of any of the above methods involving administration of a probiotic composition, the probiotic composition is directly or indirectly delivered to the digestive tract of the subject. In one embodiment, the probiotic composition is administered to the subject by a route selected from the group consisting of oral, topical, rectal (e.g., by Fecal Microbiota Transplantation (FMT), enema), mucosal, sublingual, nasal, and via naso/oro-gastric gavage. In one embodiment, the probiotic composition is delivered to the subject in a form of a liquid, foam, cream, spray, powder, or gel. In one embodiment, the probiotic composition comprises a buffering agent (e.g., sodium bicarbonate, infant formula or sterilized human milk, or other agents which allow bacteria to survive and grow (e.g., survive in the acidic environment of the stomach and to grow in the intestinal environment), along with preservatives, stabilizers, binders, compaction agents, lubricants, dispersion enhancers, disintegration agents, antioxidants, flavoring agents, sweeteners, and coloring agents.

In one embodiment of any of the above methods involving administration of a probiotic composition, the probiotic composition is administered conjointly with a prebiotic that stimulates growth and/or activity of bacteria contained in the probiotic composition. Non-limiting examples of useful prebiotics include, e.g., fructooligosaccharides (FOS), galactooligosaccharides (GOS), human milk oligosaccharides (HMO), Lacto-N-neotetraose, D-Tagatose, xylo-oligosaccharides (XOS), arabinoxylan-oligosaccharides (AXOS), N-acetylglucosamine, N-acetylgalactosamine, glucose, arabinose, maltose, lactose, sucrose, cellobiose, amino acids, alcohols, resistant starch (RS), and any mixtures thereof. In one specific embodiment, the probiotic and prebiotic are administered in one composition, or simultaneously as two separate compositions, or sequentially.

In some embodiments, a dosage for an individual comprises a predetermined quantity of the microbe calculated in an amount sufficient to produce the desired effect. The actual dosage forms will depend on the particular bacteria employed and the effect to be achieved. The composition comprising the desired microbe(s) can be administered alone or in combination with one or more additional probiotic, neutraceutical, or therapeutic agents. Administration “in combination with” one or more further additional probiotic, neutraceutical, or therapeutic agents includes both simultaneous (at the same time) and consecutive administration in any order. Administration can be chronic or intermittent, as deemed appropriate by the supervising practitioner, including in view of any change in any undesirable side effects.

The composition can be formulated as a frozen composition, e.g., flash frozen, dried or lyophilized for storage and/or transport. In addition, the composition can administered alone or in combination with a carrier, such as a pharmaceutically acceptable carrier or a biocompatible scaffold. Compositions of the disclosure may be conventionally administered rectally as a suppository, parenterally, by injection, for example, intravenously, subcutaneously, or intramuscularly. Additional formulations that are suitable for other modes of administration include oral formulations. Oral formulations include such normally employed excipients such as, for example, pharmaceutical grades of mannitol, lactose, starch, magnesium stearate, sodium saccharine, cellulose, magnesium carbonate and the like. These compositions take the form of solutions, suppositories, suspensions, tablets, pills, capsules, sustained release formulations or powders and contain about 10% to about 95% of active ingredient, preferably about 25% to about 70%.

Typically, compositions are administered in a manner compatible with the dosage formulation, and in such amount as will be therapeutically effective for the individual being treated. The quantity to be administered depends on the individual to be treated. Precise amounts of the composition to be administered depend on the judgment of the practitioner. Suitable regimes for initial administration and boosters are also variable, but are typified by an initial administration followed by subsequent administrations.

In many instances, it will be desirable to have multiple administrations of the compositions about, at most about or at least about 3, 4, 5, 6, 7, 8, 9, 10 days or more. In specific cases, the administration occurs prior to, during, and/or following the CAR T-cell therapy. The administrations will normally range from 2 day to twelve week intervals, more usually from one to two week intervals. Periodic boosters at particular intervals may be desirable to maintain the condition of the immune system.

The probiotic will be pharmaceutically acceptable or pharmacologically acceptable. The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The carrier may be a solvent or dispersion medium containing, for example, water, ethanol, polyol (for example, glycerol, propylene glycol, and liquid polyethylene glycol), and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of undesirable microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

An effective amount of the probiotic composition may be determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

3. Adoptive Cell Therapy and Chimeric Antigen Receptors

In particular embodiments of the disclosure, an individual is in need of adoptive cell therapy, including CAR T-cell therapy. The individual in particular embodiments has cancer and is in need of adoptive cell therapy that targets one or more antigens on cancer cells in the body of the individual. The individual is subject to analysis of the gut microbiome as either routine health care and such analysis is applied to cancer treatment upon need, or an individual is subject to analysis of the gut microbiome following a cancer diagnosis and in need of treatment thereof. The individual may be screened for the suitability of adoptive cell therapy by screening for the composition of the gut microbiome. In some cases, the analysis of the gut microbiome of the individual provides for determination of a treatment outcome, including whether or not the individual may receive the adoptive cell therapy with or without therapeutic intervention, in at least some cases. For example, the analysis of the gut microbiome may determine whether or not one or any adoptive cell therapies would be efficacious for the individual. If the adoptive cell therapy would be considered toxic and/or ineffective for the individual, then the adoptive cell therapy may be avoided for the individual, may be altered for the individual, or the treatment regimen may include one or more additional agents to enhance and/or render more safe the adoptive cell therapy.

In particular embodiments, the gut microbiome is analyzed for the suitability of adoptive cell transfer therapy that comprises particular immune cells that express one or more engineered antigen receptors. In particular cases, the immune cells are T-cells, NK cells, NKT cells, gamma-delta T cells, macrophages, B cells, or a mixture of these.

The adoptive cell therapy may be of any kind, but in specific embodiments the adoptive cell therapy comprises a plurality of engineered immune cells for example, that are engineered because they have been manipulated to express one or more non-natural engineered antigen receptors. Although the engineered antigen receptor may be one or more chimeric antigen receptors (CAR), one or more engineered T-cell receptors, or both, in specific embodiments the engineered antigen receptor comprises a CAR that is manipulated to comprise an antigen binding domain (such as an scFv) that targets a particular cancer antigen on cancer cells of the individual. That is, in specific embodiments the CAR is tailored or selected specifically because it targets an antigen on cancer cells of the individual. In specific embodiments, the CAR comprises two or more antigen binding domains that allow targeting of two or more corresponding cancer antigens.

In some embodiments, the CAR comprises: a) one or more intracellular signaling domains, b) a transmembrane domain, and c) an extracellular domain comprising one or more antigen binding regions. In some embodiments, the CAR comprises a transmembrane domain and one or more costimulatory domains, such as one or more of CD28, CD27, OX-40 (CD134), DAP10, DAP12, and 4-1BB (CD137). The CAR may also comprise CD3zeta, in specific embodiments.

In some embodiments, the CAR is constructed with a specificity for a particular antigen (or marker or ligand), such as an antigen expressed in a particular cell type to be targeted by adoptive therapy, e.g., a cancer marker. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb). Any suitable antigen may be targeted in the present method. The antigen may be associated with certain cancer cells but not associated with non-cancerous cells, in some cases. Exemplary antigens include, but are not limited to, tumor-/cancer-associated antigens, tumor neoantigens, antigenic molecules from infectious agents, or auto-/self-antigens.

In alternative embodiments, the adoptive cell therapy comprises an engineered antigen receptor that is not a CAR but is instead a non-native T-cell receptor. Thus, in some embodiments, the engineered heterologous antigen receptors include recombinant TCRs and/or TCRs cloned from naturally occurring T cells. A “T cell receptor” or “TCR” refers to a molecule that contains a variable α and β chains (also known as TCRα and TCRβ, respectively) or variable γ and δ chains (also known as TCRγ and TCRδ, respectively) and that is capable of specifically binding to an antigen peptide bound to a MHC receptor. In some embodiments, the TCR is in the αβ form.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex. In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the adoptive cell transfer therapy, including CAR T-cells, comprises one or more safety switches that allow the adoptive cells to be killed in the event that they are toxic to the individual. In specific embodiments, the cells comprise a vector comprising a suicide gene that will allow killing of the adoptive cells upon delivery of an agent. As one example, the suicide gene is EGFRt that is targeted by the antibody cetuximab. Another example is iCaspase9+ and AP1903. Other suicide switches include the switch HSV-TK and the switch CD20.

### C. Examples of Methods of Analysis of the Gut Microbiome

The analysis of the gut microbiome to determine its content may be performed by any suitable method. The analysis may begin with collection of a suitable sample, such as stool, tissue biopsy, or a combination thereof. In specific cases wherein stool is the sample of choice, one may collect the whole stool, homogenize it immediately (e.g., with a blender or a tissue homogenizer), then flash freeze the homogenate in liquid nitrogen or in dry ice/ethanol slurry, with an aliquot preserved in a certain percentage of glycerol in suitable media for culturing. The individual that obtains the sample may or may not be the individual that performs the analysis. In some cases, the sample is stored prior to analysis, whereas in other cases the sample is analyzed without storage.

In particular embodiments, the gut microbiome is analyzed based on shotgun sequencing of nucleic acid of the microbe(s), including shotgun metagenomics sequencing, such as to provide more in-depth reads. In specific embodiments, the majority or substantially all of the genomic DNA for a microbe is analyzed instead of a specific region of DNA. However, in certain embodiments, analysis of a specific region of DNA is utilized, such as with 16S rRNA sequencing.

Other analysis methods may be utilized, either alone or with other methods. As one example, for known organisms with well-characterized selective culture conditions, culturing may be utilized as a detection method. Assay panels that target a set of known microbes or genes thereof may be utilized. Stool samples may be processed through nucleic acid extraction followed by complementary DNA synthesis and subsequent amplification using mixtures of primers specific for a given range of organisms. Either genomic DNA or PCR product may then be qualified and quantified, such as through a hybridization array using a fluorescence-based measure or a melt curve analysis. In specific embodiments, quantitative PCR and reverse-transcription quantitative PCR may be utilized.

In particular embodiments, amplicon analyses are employed in which a specific region of DNA is amplified by orders of magnitude using various methods including PCR. In specific cases, the PCR primers match a specific region, such as the 16S rRNA for bacteria. Bacterial 16S rRNA genes contain 9 hypervariable regions (V1-V9) that show sequence diversity and can be used as a barcode-like method to differentiate many bacterial taxa, including at the species level. In some cases, next-generation sequencing may be performed to read the sequences. In other cases, instead of using one gene, such as 16S rRNA, shotgun metagenomics is utilized that fragments all the DNA from a sample into small pieces, sequences these fragments, and then the sequenced fragments are arranged accordingly to provide information on a grander scale for the microbe identification.

In particular cases, fecal baseline samples were collected from lymphoma patients. In brief, genomic DNA was isolated using QIAamp DNA stool mini kit (Qiagen), according to the manufacturer's protocol, modified to include an intensive bead-beating lysis step. The V4 region of 16S rRNA gene was amplified by PCR from 10 ng of each of extracted and purified genomic DNA using 515 forward and 806 reverse primer pairs (Caporaso, J. G. et al. ISME J. 6, 1621-1624 2012). The amplicon pool was purified with QIAquick gel extraction kit (Qiagen) and sequenced on the Illumina Miseq sequencer platform using 2×250 bp paired-end protocol.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the subject matter of the disclosure.

### Example 1

Gut Microbiome as a Predictive Biomarker of Outcomes after Car T-Cell Therapy and its Modulation to Enhance Car-T Efficacy and Reduce Toxicity

Baseline stool samples were collected from relapsed/refractory large B-cell lymphoma patients undergoing treatment with anti-CD19 CAR-T therapy. Taxonomic profiling was performed on all samples using targeted ribosomal 16S RNA gene sequencing of the V4 region and also, calculated various microbiome diversity metrics to determine association with response, survival outcomes, and toxicities.

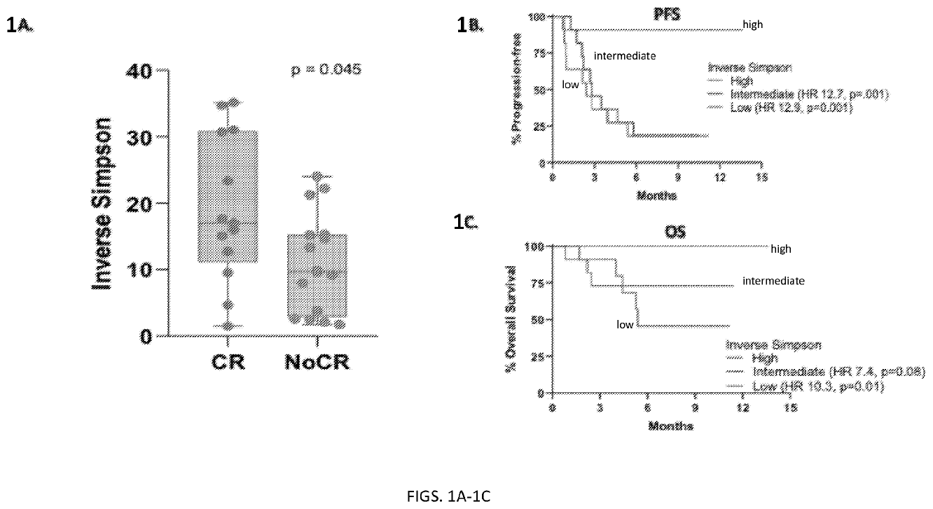

Patients with high microbial diversity as measured by Inverse Simpson Index (ISI) correlated significantly with progression free survival and overall survival as shown in the figure below. Since ongoing response at 3 months was previously shown to be associated with long-term durability (Locke et al, Lancet Oncol 2019), we analyzed differences in baseline gut microbiome markers in patients with or without ongoing complete response (CR) at 3 months post-CAR-T infusion. Patients with ongoing complete response at 3 months had significantly higher ISI compared to patients who did not as shown in FIG. 1A. We also analyzed the impact of gut microbial diversity on progression-free (PFS) and overall survival (OS) by stratifying the patients according to the tertile of ISI. The PFS of patients in the highest tertile of ISI values (n=11, median PFS not reached) was significantly higher compared to those with intermediate (n=11, median PFS=2.82 months, HR 12.7, 95% CI 3.61 to 44.77, log-rank, p=0.001) or low (n=11, median PFS=2.43 months, HR 12.9, 95% CI 3.68 to 45.75, log-rank, p=0.001) ISI values (FIG. 1B). High ISI values also correlated positively and significantly with OS (FIG. 1C).

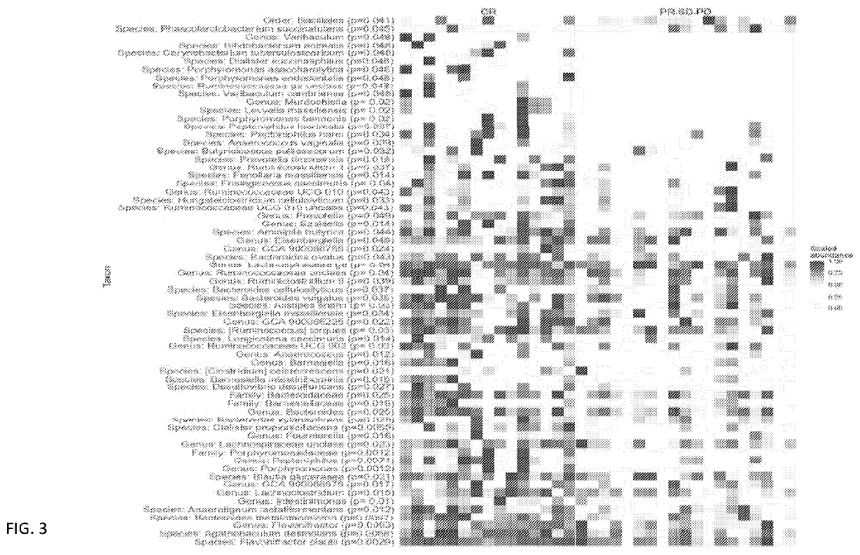

There was an increased relative abundance of several bacterial families in patients with ongoing CR versus those who did not (partial response, PR; stable disease, SD; and progressive disease, PD). Some of the example of these species include Genus Falvonifractor, genus Intestimonas, genus Lachnoclostridium, genus peptoniphilus etc. The list of the bacteria associating with responses is provided in FIG. 2 and FIG. 3.

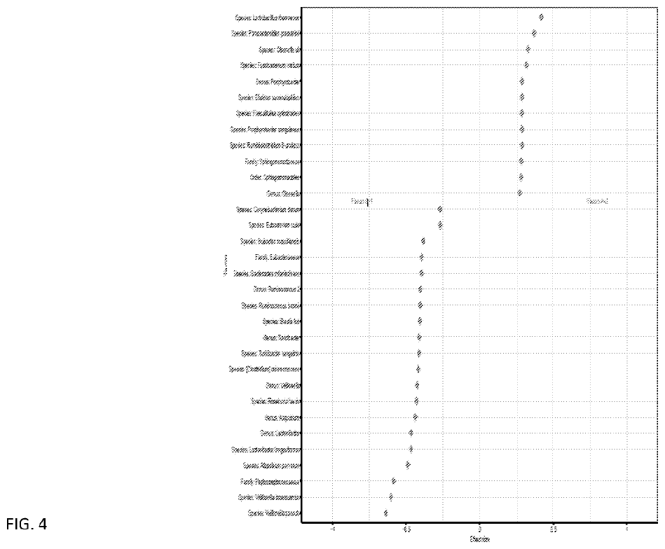

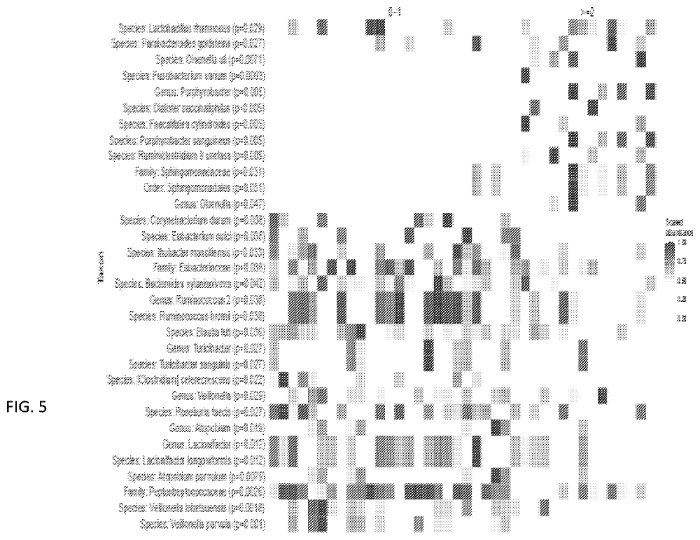

There was a difference in microbial composition associating with toxicities related to CAR-T therapy such as cytokine release syndrome (CRS) and immune effector cell-associated neurotoxicity syndrome (ICANS) as shown in figures below. For example, species Veillonella parvula, genus Lactonifactor, genus Atopobium, genus ruminococcus, etc., are associated with lower grade of CRS, as shown in FIG. 4 and FIG. 5.

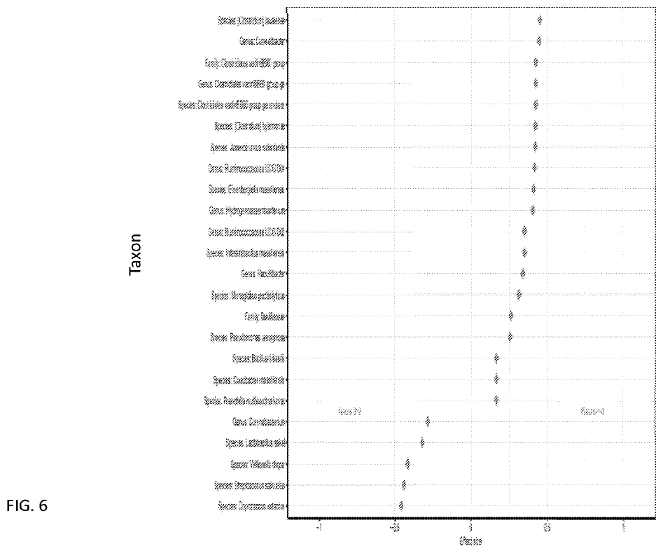

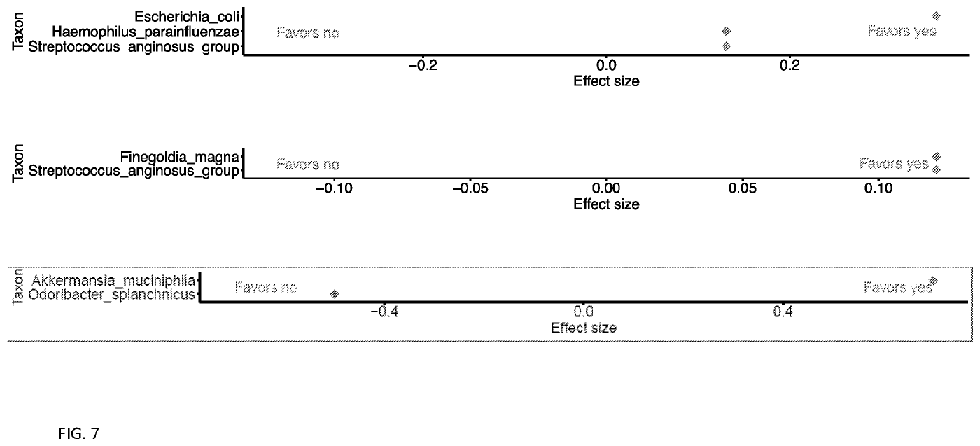

Similarly, few species such as genus Cuneatibacter, genus Clostridiales, enus Raolutibacter associated with higher grade of ICANS as shown in FIG. 6. FIG. 7 demonstrates analysis of a variety of bacteria to indicate whether or not their presence favors disease progression. Five non-efficacious bacteria groups included Finegoldia magna, Streptococcus anginosus group, Akkermansia muciniphila, Escherichia coli, and Haemophilus parainfluenzae. One bacteria was efficacious by not favoring disease progress: Odoribacter splanchnicus.

In particular embodiments, gut microbiome diversity metrics is a strong predictor of durability of responses and survival after CAR T-cell therapy. In addition, differences in gut bacterial composition and abundance influence responses and toxicities associated with CAR-T-therapy. The modulation of the gut microbiome has significant potential for influencing efficacy and toxicity for CAR T-cell therapy.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of determining or predicting a therapy response for an individual, comprising the step of analyzing a microbe composition from the gut microbiome of the individual, wherein:
(a) the therapy for the individual will not be efficacious or has a risk of not being efficacious, compared to a standard or another individual, when the gut microbiome for the individual comprises, consists of, or consists essentially of one or more microbes from the Order Bacillales; and/or the species Phascolarctobacterium succinatutens; and/or Finegoldia magna; and/or Streptococcus anginosus group; and/or Akkermansia muciniphila; and/or Escherichia coli; and/or Haemophilus parainfluenzae; and/or
(b) the therapy for the individual will be efficacious or has an increased chance of being efficacious, compared to a standard or another individual, when the gut microbiome for the individual comprises, consists of, or consists essentially of one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii.

2. The method of claim 1(a), wherein the individual has cancer and the response to the therapy for the individual is or has a risk of being a partial response, stable disease, or progressive disease.

3. The method of claim 1(b), wherein the individual has cancer and the response to the therapy for the individual is or has an increased chance of being a complete response.

4. The method of claim 2 or 3, wherein the cancer comprises a solid tumor or is a hematological malignancy.

5. The method of claim 1(b), wherein the gut microbiome of the individual comprises Flavonifractor plautii.

6. The method of any one of claims 1-5, wherein the therapy comprises immunotherapy.

7. The method of claim 6, wherein the immunotherapy comprises adoptive cell therapy.

8. The method of claim 7, wherein the adoptive cell therapy comprises adoptive T-cell therapy.

9. The method of claim 7 or 8, wherein the cells of the adoptive cell therapy are modified to comprise one or more engineered antigen receptors.

10. The method of claim 9, wherein the engineered antigen receptor comprises one or more chimeric antigen receptors (CAR) or one or more non-native T-cell receptors or the cells have one or more of both.

11. The method of any one of claims 7-10, wherein the adoptive cell therapy comprises CAR T-cell therapy.

12. The method of claim 1(b), further comprising the step of administering a therapeutically effective amount of the therapy to the individual.

13. The method of claim 1(a), wherein the therapy is CAR T-cell therapy and the CAR T-cell therapy is modified prior to administering to the individual to enhance efficacy of the CAR T-cell therapy.

14. The method of claim 13, wherein the dosage of the CAR T-cell therapy is increased.

15. The method of claim 13 or 14, wherein one or more components of the CAR are altered.

16. The method of any one of claims 1-15, wherein the individual is administered an effective amount of one or more probiotics and/or one or more fecal transplants.

17. The method of claim 16, wherein the one or more probiotics and/or one or more fecal transplants comprises, consists of, or consists essentially of one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii.

18. The method of any one of claims 1-17, wherein the individual is provided a therapeutically effective amount of another cancer therapy.

19. A method of determining or predicting toxicity of a therapy for an individual, comprising the step of analyzing a microbe composition from the gut microbiome of the individual, wherein:
(a) the therapy will be toxic for the individual, or has a risk of being toxic for the individual, compared to a standard or another individual, when the gut microbiome comprises, consists of, or consists essentially of one or more of Species Lactobacillus rhamnosus; Species Parabacteroides goldsteinii; Species Olsenella uli; Species Fusobacterium varium; Genus Porphyrobacter; Species Dialister succinatiphilus; Species Faecalitalea cylindroides; Species Porphyrobacter sanguineus; Species Ruminiclostridium 9 unclass; Family Sphingomonadaceae; Order Sphingomonadales; and Genus Olsenella; and/or
(b) the therapy will not be toxic for the individual or has an increased likelihood of not being toxic for the individual, compared to a standard or another individual, when the gut microbiome comprises, consists of, or consists essentially of one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula.

20. The method of claim 19, wherein the toxicity comprises cytokine release syndrome (CRS).

21. The method of claim 20, wherein determining the toxicity is further defined as determining a grade of toxicity associated with CRS.

22. The method of any one of claims 19-21, wherein the therapy comprises immunotherapy.

23. The method of claim 22, wherein the immunotherapy comprises adoptive cell therapy.

24. The method of claim 23, wherein the adoptive cell therapy comprises adoptive T-cell therapy.

25. The method of claim 23 or 24, wherein the cells of the adoptive cell therapy are modified to comprise one or more engineered antigen receptors.

26. The method of claim 25, wherein the engineered antigen receptor comprises one or more chimeric antigen receptors (CAR) or one or more non-native T-cell receptors or the cells have one or more of both.

27. The method of any one of claims 23-26, wherein the adoptive cell therapy comprises CAR T-cell therapy.

28. The method of claim 19(b), further comprising the step of administering a therapeutically effective amount of the therapy to the individual.

29. The method of claim 19(a), wherein the therapy is CAR T-cell therapy and the CAR T-cell therapy is modified prior to administering to the individual to reduce toxicity of the CAR T-cell therapy.

30. The method of claim 29, wherein the dosage of the CAR T-cell therapy is decreased.

31. The method of claim 29 or 30, wherein one or more components of the CAR are altered.

32. The method of any one of claims 19-31, wherein the individual is administered an effective amount of one or more probiotics and/or one or more fecal transplants.

33. The method of claim 32, wherein the one or more probiotics and/or one or more fecal transplants comprises, consists of, or consists essentially of one or more microbes from Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula.

34. The method of any one of claims 19-33, wherein the individual is provided a therapeutically effective amount of another cancer therapy.

35. A method of determining the likelihood of an individual receiving an immunotherapy to have a particular grade of immune effector cell-associated neurotoxicity syndrome (ICANS) toxicity following receipt of the immunotherapy, comprising the step of analyzing the gut microbiome of the individual, wherein:
(a) the individual is likely of having or at risk for having Grade 3 or 4 of ICANS toxicity when the gut microbiome comprises, consists of, or consists essentially of one or more of Species [Clostridium] lavalense; Genus Cuneatibacter; Family Clostridiales vadin BB60 group; Genus Clostridiales vadin BB60 group; Species Clostridiales vadin BB60 group ge unclass; Species [Clostridium] hylemonae; Species Anaerotruncus rubiinfantis; Genus Ruminococcaceae UCG 004; Species Eisenbergiella massiliensis; Genus Hydrogenoanaerobacterium; Genus Ruminococcaceae UCG 008; Species Intestinibacillus massiliensis; Genus Raoultibacter; Species Monoglobus pectinilyticus; Family Bacillaceae; Species Pseudomonas aeruginosa; Species Bacillus hisashii; Species Caecibacter massiliensis; and Species Prevotella multisaccharivorax; and/or
(b) the individual is likely of having or at risk for having Grade 1 or 2 of ICANS toxicity when the gut microbiome comprises, consists of, or consists essentially of one or more of Genus Corynebacterium; Species Lactobacillus sakei; Species Veillonella dispar; Species Streptococcus salivarius; and Species Coprococcus eutactus.

36. The method of claim 35, wherein the therapy comprises immunotherapy.

37. The method of claim 36, wherein the immunotherapy comprises adoptive cell therapy.

38. The method of claim 37, wherein the adoptive cell therapy comprises adoptive T-cell therapy.

39. The method of claim 37 or 38, wherein the cells of the adoptive cell therapy are modified to comprise one or more engineered antigen receptors.

40. The method of claim 39, wherein the engineered antigen receptor comprises one or more chimeric antigen receptors (CAR) or one or more non-native T-cell receptors or the cells have one or more of both.

41. The method of any one of claims 37-40, wherein the adoptive cell therapy comprises CAR T-cell therapy.

42. The method of claim 35(b), further comprising the step of administering a therapeutically effective amount of the immunotherapy to the individual.

43. The method of claim 35(a), wherein following the analysis the immunotherapy is modified to reduce the risk of ICANS prior to delivery to the individual.

44. The method of claim 35(a), wherein the therapy is CAR T-cell therapy and the CAR T-cell therapy is modified prior to administering to the individual to reduce toxicity of the CAR T-cell therapy.

45. The method of claim 44, wherein the dosage of the CAR T-cell therapy is decreased.

46. The method of claim 44 or 45, wherein one or more components of the CAR are altered.

47. The method of any one of claims 35-46, wherein the individual is administered an effective amount of one or more probiotics and/or one or more fecal transplants.

48. The method of claim 47, wherein the one or more probiotics and/or one or more fecal transplants comprises, consists of, or consists essentially of one or more of Genus Corynebacterium; Species Lactobacillus sakei; Species Veillonella dispar; Species Streptococcus salivarius; and Species Coprococcus eutactus.

49. The method of any one of claims 35-48, wherein the individual is provided a therapeutically effective amount of another cancer therapy.

50. A method of treating cancer in an individual, comprising the step of providing a therapeutically effective amount of an immunotherapy to the individual when:
(a) the gut microbiome of the individual comprises, consists of, or consists essentially of one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) the gut microbiome of the individual comprises, consists of, or consists essentially of one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) the gut microbiome of the individual comprises, consists of, or consists essentially of one or more of Genus Corynebacterium; Species Lactobacillus sakei; Species Veillonella dispar; Species Streptococcus salivarius; and Species Coprococcus eutactus.

51. The method of claim 50, wherein the immunotherapy comprises adoptive cell therapy.

52. The method of claim 51, wherein the adoptive cell therapy comprises adoptive T-cell therapy.

53. The method of claim 51 or 52, wherein the cells of the adoptive cell therapy are modified to comprise one or more engineered antigen receptors.

54. The method of claim 53, wherein the engineered antigen receptor comprises one or more chimeric antigen receptors (CAR) or one or more non-native T-cell receptors or the cells have one or more of both.

55. The method of any one of claims 50-54, wherein the adoptive cell therapy comprises CAR T-cell therapy.

56. The method of any one of claims 50-55, wherein the individual is administered an effective amount of one or more probiotics and/or one or more fecal transplants.

57. The method of claim 56, wherein the one or more probiotics and/or one or more fecal transplants comprises, consists of, or consists essentially of one or more microbes of any one or more of (a), (b), and/or (c):
(a) one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) one or more of Genus Corynebacterium; Species Lactobacillus sakei; Species Veillonella dispar; Species Streptococcus salivarius; and Species Coprococcus eutactus.

58. The method of any one of claims 50-57, wherein the individual is provided a therapeutically effective amount of another cancer therapy.

59. A method of determining a therapy outcome for an individual in need of adoptive cell therapy, comprising the step of analyzing the gut microbiome for diversity of microbes therein, wherein when the gut microbiome of the individual has high diversity, the individual has an increased likelihood of efficacious adoptive cell therapy, compared to an individual that lacks high diversity of the gut microbiome.

60. The method of claim 59, wherein when the individual has a low diversity of microbes in the gut microbiome, the individual is provided an effective amount of one or more fecal transplantations and/or one or more probiotic compositions.

61. The method of claim 59 or 60, wherein the diversity of the gut microbiome is determined by Inverse Simpson Index (ISI).

62. The method of claim 61, wherein when the diversity of the individual's gut microbiome is in the highest tertile as determined by ISI, the individual has an increased likelihood of efficacious adoptive cell therapy.

63. The method of any one of claims 59-62, wherein the identity of one or more microbes in the microbiome is determined by shotgun sequencing of the genome of the one or more microbes.

64. The method of any one of claims 59-62, wherein the identity of one or more microbes in the microbiome is determined by directed sequencing of the genome of the one or more microbes.

65. The method of claim 64, wherein the directed sequencing is of 16S rRNA of the one or more microbes.

66. The method of any one of claims 62-65, wherein when the diversity of the individual's gut microbiome is in the highest tertile as determined by ISI, the individual is administered an effective amount of the adoptive cell therapy.

67. The method of claim 66, wherein the adoptive cell therapy is CAR T-cell therapy.

68. The method of any one of claims 62-65, wherein when the diversity of the individual's gut microbiome is not in the highest tertile as determined by ISI, the individual is not administered an effective amount of the adoptive cell therapy.

69. The method of any one of claims 62-65, wherein the adoptive cell therapy is CAR T-cell therapy and wherein when the diversity of the individual's gut microbiome is not in the highest tertile as determined by ISI, the CAR T-cell therapy is modified.

70. The method of claim 69, wherein the dosage of the CAR T-cell therapy is decreased.

71. The method of claim 69 or 70, wherein one or more components of the CAR are altered.

72. The method of any one of claims 66-71, wherein the individual is administered an effective amount of one or more probiotics and/or one or more fecal transplants.

73. The method of claim 72, wherein the one or more probiotics and/or one or more fecal transplants comprises, consists of, or consists essentially of one or more microbes of any one or more of (a), (b), and/or (c):
(a) one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) one or more of Genus Corynebacterium; Species Lactobacillus sakei; Species Veillonella dispar; Species Streptococcus salivarius; and Species Coprococcus eutactus.

74. A probiotic composition and/or fecal transplant composition, comprising, consisting of, or consisting essentially of one or more microbes of any one or more of (a), (b), (c) and/or (d):
(a) one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii;
(b) one or more of Species Corynebacterium durum; Species Eubacterium sulci; Species Ihubacter massiliensis; Family Eubacteriaceae; Species Bacteroides xylanisolvens; Genus Ruminococcus 2; Species Ruminococcus bromii; Species Blautia luti; Genus Turicibacter; Species Turicibacter sanguinis; Species [Clostridium] celerecrescens; Genus Veillonella; Species Roseburia faecis; Genus Atopobium; Genus Lactonifactor; Species Lactonifactor longoviformis; Species Atopobium parvulum; Family Peptostreptococcaceae; Species Veillonella tobetsuensis; and Species Veillonella parvula; and/or
(c) one or more of Genus Corynebacterium; Species Lactobacillus sakei; Species Veillonella dispar; Species Streptococcus salivarius; and Species Coprococcus eutactus; and/or
(d) Odoribacter splanchnicus.

75. A method of determining or predicting a therapy response for an individual, comprising the step of analyzing a microbe composition from the gut microbiome of the individual, wherein:
(a) the therapy for the individual will not be efficacious or has a risk of not being efficacious, compared to a standard or another individual, when the gut microbiome for the individual comprises, consists of, or consists essentially of one or more microbes from Finegoldia magna; and/or Streptococcus anginosus group; and/or Akkermansia muciniphila; and/or Escherichia coli; and/or Haemophilus parainfluenzae; and/or
(b) the therapy for the individual will be efficacious or has an increased chance of being efficacious, compared to a standard or another individual, when the gut microbiome for the individual comprises, consists of, or consists essentially of Odoribacter splanchnicus.

76. The method of claim 75(a), wherein the individual has cancer and the response to the therapy for the individual is or has a risk of being a partial response, stable disease, or progressive disease.

77. The method of claim 75(b), wherein the individual has cancer and the response to the therapy for the individual is or has an increased chance of being a complete response.

78. The method of claim 76 or 77, wherein the cancer comprises a solid tumor or is a hematological malignancy.

79. The method of any one of claims 75-78, wherein the therapy comprises immunotherapy.

80. The method of claim 79, wherein the immunotherapy comprises adoptive cell therapy.

81. The method of claim 80, wherein the adoptive cell therapy comprises adoptive T-cell therapy.

82. The method of claim 80 or 81, wherein the cells of the adoptive cell therapy are modified to comprise one or more engineered antigen receptors.

83. The method of claim 82, wherein the engineered antigen receptor comprises one or more chimeric antigen receptors (CAR) or one or more non-native T-cell receptors or the cells have one or more of both.

84. The method of any one of claims 80-83, wherein the adoptive cell therapy comprises CAR T-cell therapy.

85. The method of claim 75(b), further comprising the step of administering a therapeutically effective amount of the therapy to the individual.

86. The method of claim 75(a), wherein the therapy is CAR T-cell therapy and the CAR T-cell therapy is modified prior to administering to the individual to enhance efficacy of the CAR T-cell therapy.

87. The method of claim 86, wherein the dosage of the CAR T-cell therapy is increased.

88. The method of claim 86 or 87, wherein one or more components of the CAR are altered.

89. The method of any one of claims 75-88, wherein the individual is administered an effective amount of one or more probiotics and/or one or more fecal transplants.

90. The method of claim 89, wherein the one or more probiotics and/or one or more fecal transplants comprises, consists of, or consists essentially of Odoribacter splanchnicus and/or one or more microbes from Genus Varibaculum; Species Bifidobacterium animalis; Species Corynebacterium tuberculostearicum; Species Dialister succinatiphilus; Species Porphyromonas asaccharolytica; Species Porphyromonas endodontalis; Species Ruminococcaceae ge unclass; Species Varibaculum cambriense; Genus Murdochiella; Species Levyella massiliensis; Species Porphyromonas bennonis; Species Peptoniphilus lacrimalis; Species Peptoniphilus harei; Species Anaerococcus vaginalis; Species Butyricicoccus pullicaecorum; Species Prevotella timonensis; Genus Ruminiclostridium 1; Species Fenollaria massiliensis; Species Frisingicoccus caecimuris; Genus Ruminococcaceae UCG 010; Species Hungateiclostridium cellulolyticum; Species Ruminococcaceae UCG 010 unclass; Genus Prevotella; Genus Ezakiella; Species Aminipila butyrica; Genus Eisenbergiella; Genus GCA 900066755; Species Bacteroides ovatus; Genus Lachnospiraceae ge; Genus Ruminococcaceae unclass; Genus Ruminiclostridium 9; Species Bacteroides cellulosilyticus; Species Bacteroides vulgatus; Species Alistipes shahii; Species Eisenbergiella massiliensis; Genus GCA 900066225; Species [Ruminococcus] torques; Species Longicatena caecimuris; Genus Ruminococcaceae UCG 002; Genus Anaerococcus; Genus Barnesiella; Species [Clostridium] celerecrescens; Species Barnesiella intestinihominis; Species Desulfovibrio desulfuricans; Family Bacteroidaceae; Family Barnesiellaceae; Genus Bacteroides; Species Bacteroides xylanisolvens; Species Dialister propionicifaciens; Genus Fournierella; Genus Lachnospiraceae unclass; Family Porphyromonadaceae; Genus Peptoniphilus; Genus Porphyromonas; Species Blautia glucerasea; Genus GCA 900066575; Genus Lachnoclostridium; Genus Intestinimonas; Species Anaerotignum lactatifermentans; Species Bacteroides thetaiotaomicron; Genus Flavonifractor; Species Agathobaculum desmolans; and Species Flavonifractor plautii.

91. The method of any one of claims 75-90, wherein the individual is provided a therapeutically effective amount of another cancer therapy.
